(19) United States
(12) Patent Application Publication
(10) Pub. No.: US 2009/0215896 A1
(43) Pub. Date: Aug. 27, 2009

(54) REELIN DEFICIENCY OR DYSFUNCTION AND METHODS RELATED THERETO

(75) Inventors: John P. Morseman, Columbia, MD (US); Mark W. Moss, Baltimore, MD (US); Lorie A. Ellis, BelAir, MD (US)

Correspondence Address: SHERIDAN ROSS P.C., 1560 BROADWAY, SUITE 1200, DENVER, CO, 80202, US

(73) Assignee: MARTEK BIOSCIENCES CORPORATION, Columbia, MD (US)

(21) Appl. No.: 10/597,304

(22) PCT Filed: Jan. 19, 2005
(86) PCT No.: PCT/US05/02177

§ 371 Date: Jul. 19, 2006

Related U.S. Application Data
(60) Provisional application No. 60/537,600, filed on Jan. 19, 2004.
(60) Provisional application No. 60/605,219, filed on Aug. 27, 2004.

(51) Int. Cl. A61K 31/201; G01N 33/543; C40B 30/04; C12Q 1/02; C12Q 1/68; C07K 14/47

(52) U.S. Cl. 514/560; 436/518; 506/9; 435/29; 435/6; 530/350

(57) ABSTRACT

A method of measuring Reelin as a biomarker, to non-destructively assess or predict DHA levels in the brain and in other, currently inaccessible or difficult-to-access, key components of the central nervous system (CNS) is described. Also described is a method to prevent, delay the onset of, or treat Reelin deficiency or dysfunction and/or a disease or condition associated with Reelin deficiency or dysfunction, comprising administering to a patient diagnosed with or suspected of having a Reelin deficiency or dysfunction an amount of a PUFA, and particularly an omega-3 PUFA, and more particularly, docosahexaenoic acid (DHA) or a precursor or source thereof, to compensate for the effects of Reelin deficiency or dysfunction in the patient. Also described is a method to prevent or reduce developmental defects or disorders associated with Reelin dysfunction or deficiency through the supplemental use of polyunsaturated fatty acids (PUFAs—unsaturated fatty acids having two or more double bonds), and particularly highly unsaturated fatty acids (HUFAs—unsaturated fatty acids having three or more double bonds), and more particularly a HUFA selected from arachidonic acid (ARA), eicosapentacnoic acid (EPA), docosahexaenoic acid (DHA) and docosapentaenoic acid (DPA), and even more particularly omega-3 HUFAs, and more particularly DHA, to: compensate for reduced fatty acid binding protein or function thereof in the patient; compensate for reduced brain lipid binding protein or function thereof in the patient; improve the activity of fatty acid binding proteins in the patient; increase the expression of brain lipid binding proteins (BLBPs) in the patient; improve at least one parameter of the mechanism of action of brain lipid binding proteins in the patient; overcome a deficiency of DHA in central nervous system (CNS) structures and improve the resulting function thereof; increase the incorporation of functional DHA and other PUFAs into the phospholipid membranes of glial cells and neurons in the patient; increase the level of Reelin and/or improve the activity of Reelin in the patient; and/or improve at least one symptom of a disease or condition associated with Reelin deficiency or dysfunction.

### FIELD OF THE INVENTION

The present invention generally relates to methods of treating Reelin deficiency or dysfunction and conditions or disorders associated therewith through the supplemental use of agents that have a high affinity for brain lipid binding proteins, (BLBPs), and particularly omega-3 and/or omega-6 polyunsaturated fatty acids (PUFAs), such as docosahexaenoic acid (DHA 22:6 n-3). The present invention also relates to the use of Reelin as a biomarker for DHA and other PUFA levels in the brain and other tissues.

### BACKGROUND OF THE INVENTION

Neurological or neuropsychiatric disorders and diseases have continually been a challenge to predict, identify and diagnose. The cause of some of the more significant neurodegenerative abnormalities (e.g., schizophrenia, bipolar disorder, dyslexia, dyspraxia, attention deficit hyperactivity disorder (ADHD), epilepsy, autism, Parkinson's Disease, senile dementia, Alzheimer's Disease, peroxisomal proliferator activation disorder (PPAR), multiple sclerosis, diabetes-induced neuropathy, macular degeneration, retinopathy of prematurity, Huntington's Disease, amyotrophic lateral sclerosis (ALS), retinitis pigmentosa, cerebral palsy, muscular dystrophy, cancer, cystic fibrosis, neural tube defects, depression, Zellweger syndrome, Lissencepahly, Down's Syndrome, Muscle-Eye-Brain Disease, Walker-Warburg Syndrome, Charoct-Marie-Tooth Disease, inclusion body myositis (IBM) and Aniridia) may partially be from a dysfunction in neuronal migration or neuronal positioning in the brain.

Reelin, an extracellular signaling glycoprotein, plays a pivotal role in proper neuronal migration, neuronal orientation, and as a developmental regulator by maintaining the radial glial system in the central and peripheral nervous system. Reelin has also been implicated in proper lamination of neurons. During development, Reelin is found at high levels in the liver, kidney, brain, spinal cord and the retina (D'Arcangelo et al., Nature 374:719-723, 1995). However, unlike many developmental genes, Reelin continues to be expressed throughout life.

Associated with levels of Reelin in the developing brain are levels of brain lipid binding proteins (BLBP), which function as a member of the family of fatty acid binding proteins (FABP). Hartfuss et al. (Development, 2003; 130, 4597-4609) showed that the addition (in vitro) of Reelin increases the BLBP content in the cortex of the brain. Typically found in glial cells in the developing central and peripheral nervous systems, BLBP (or Brain-FABP) appears to function in the transport, deposition or protective storage of certain lipids (e.g., omega-3 fatty acids) to ensure a constant supply of fatty acids to the developing central nervous system (CNS) (Ganesaratnam K. Balendiran et al., 2000 The Journal of Biological Chemistry, 275, No. 35, 27045-27054).

One such essential omega-3 fatty acid, DHA, (4,7,10,13,16,19-docosahexaenoic acid; 22:6 n-3), is the most abundant n-3 polyunsaturated fatty acid in the brain (Williard et al., Journal of Lipid Research, 2001, 42, 1368-1376). BLBP (Brain-FABP) has a high affinity and specificity for DHA, and it is thought that BLBP may act to protect DHA from undergoing free-radical peroxidation (Ganesaratnam K. Balendiran et al., 2000).

Various levels of Reelin have been found in patients suffering from neurological disorders. For example, according to Fatemi et al. (Neuroreport 2001 Oct. 29; 12(15):3209-3215), varying reduced levels of Reelin were found in the brains of patients suffering from schizophrenia, bipolar disorder and major depression. In addition, Chen et al. (Nuclei Acids Res. 2002 Jul. 1: 30(13):2930-2939) showed that patients suffering from schizophrenia or bipolar illness with psychosis had lower than normal Reelin levels in the brain. Persico et al. (Mol Psychiatry, 2001 March; 6(2):150-159) demonstrated that autistic patients having a longer size variant of the Reelin gene (>11 GGC repeats) had lower Reelin levels in the brain and conferred a greater vulnerability to autism.

Treatment for the prevention, reduction or cure of neurological diseases or injuries traditionally focuses on a pharmaceutical approach. For example, neuropsychiatric or neurodegenerative drugs are continually being developed which alleviate symptoms, but fail to alleviate the inherent cause of the neurological problem. Thus, there is a further need in the art for novel therapeutic strategies for the treatment of neurological disorders, diseases or injuries.

### SUMMARY OF THE INVENTION

One embodiment of the invention relates to a method to treat a Reelin deficiency or dysfunction. The method includes administering to a patient diagnosed with or suspected of having a Reelin deficiency or dysfunction an amount of a polyunsaturated fatty acid (PUFA) selected from: an omega-3 PUFA and an omega-6 PUFA, or a precursor or source thereof, to compensate for the effects of Reelin deficiency or dysfunction in the patient. In one aspect the Reelin deficiency or dysfunction is associated with a decrease in the expression or function of a fatty acid binding protein (e.g., a brain lipid binding protein (BLBP)) in the patient.

Preferably, administration of the PUFA to the patient: compensates for reduced fatty acid binding protein or function thereof in the patient, compensates for reduced brain lipid binding protein or function thereof in the patient, improves the activity of fatty acid binding proteins in the patient, improves at least one parameter of the mechanism of action of brain lipid binding proteins in the patient, results in increased incorporation of functional DHA into the phospholipid membranes of glial cells and neurons in the patient, increases the level of Reelin in the patient, and/or improves the activity of Reelin in the patient.

Patients to be treated according to this method of the invention include patients suffering from or at risk of suffering from, a disease or condition associated with the Reelin deficiency or dysfunction, such that administration of the PUFA to the patient improves at least one symptom of the disease or condition, or prevents or delays the onset of the disease or condition. In one aspect, the patient has, is suspected of having, or is at risk of developing, a neurological disorder or neuropsychiatric disorder. In another aspect, the patient suffers from seizures. In another aspect, the patient has, is suspected of having, or is at risk of developing, an autoimmune disorder associated with a neurological dysfunction. In yet another aspect, the patient has an anti-phospholipid disorder. In another aspect, the patient has, is suspected of having, or is at risk of developing, a disorder selected from: schizophrenia, bipolar disorder, dyslexia, dyspraxia, attention deficit hyperactivity disorder (ADHD), epilepsy, autism, Parkinson's Disease, senile dementia, Alzheimer's Disease, peroxisomal proliferator activation disorder (PPAR), multiple sclerosis, diabetes-induced neuropathy, macular degeneration, retinopathy of prematurity, Huntington's Disease, amyotrophic lateral sclerosis (ALS), retinitis pigmentosa, cerebral palsy, muscular dystrophy, cancer, cystic fibrosis, neural tube defects, depression, Zellweger syndrome, Lissencepahly, Down's Syndrome, Muscle-Eye-Brain Disease, Walker-Warburg Syndrome, Charoct-Marie-Tooth Disease, inclusion body myositis (IBM) or Aniridia. In yet another aspect, the patient has a thyroid disorder.

In one aspect of this embodiment, prior to the step of administering, the method includes measuring an amount or a biological activity of Reelin in a biological sample from the patient. For example, the method can include comparing the amount of Reelin in the patient sample to a baseline amount of Reelin in a sample of the same type, wherein a change in the amount of Reelin in the patient sample as compared to the baseline amount indicates that the patient has a Reelin deficiency. The step of measuring can be performed by a method including, but not limited to: mRNA transcription analysis, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, or protein microchip or microarray.

In one aspect, the method can include the step of determining the relative expression or activity of different Reelin size forms in the patient to establish a Reelin size form profile in the patient sample, and comparing the patient Reelin size form profile to a baseline profile of Reelin size forms in a sample of the same type, wherein a change in expression of one or more size forms of Reelin as compared to relative expression or activity of the size forms in the baseline profile indicates that the patient has a Reelin deficiency or dysfunction. This step of measuring can be performed by a method including, but not limited to: mRNA transcription analysis, Western blot, immunoblot, and capillary electrophoresis.

In another aspect, the method can include a step of comparing the activity of Reelin in the patient sample to a baseline activity of Reelin in a sample of the same type, wherein a change in the level of activity of Reelin in the patient sample as compared to the baseline level indicates that the patient has a Reelin dysfunction. The step of measuring can be performed by a technique including, but not limited to: a receptor-ligand assay and a phosphorylation assay.

In yet another aspect, the method can include a step of measuring the levels of thyroid stimulating hormone (TSH) in the patient sample and comparing the amount of TSH in the patient sample to a baseline amount of TSH in a sample of the same type, wherein a change in the amount of TSH in the patient sample as compared to the baseline amount indicates that the patient has a TSH deficiency. In this aspect, the method may further comprise a step of administering a thyroid medication in conjunction with the PUFA, to the patient.

In the above-described methods, when a biological sample is obtained, such sample can include, but is not limited to: a cell sample, a tissue sample, and a bodily fluid sample, with a blood sample being particularly preferred.

In one aspect of this embodiment, the method can further include: monitoring the efficacy of the administration of the PUFA on Reelin levels or biological activity in the patient at least one time subsequent to the step of administering; or monitoring the efficacy of the administration of the PUFA on changes in the expression or biological activity of one or more size forms of Reelin in the patient at least one time subsequent to the step of administering. In these aspects, the method can further include a step of adjusting the administration of the PUFA to the patient in subsequent treatments based on the results of the monitoring of efficacy of the treatment.

Another embodiment of the present invention relates to a method of modulating Reelin expression in tissues or fluids. This method includes a step of administering to a patient an amount of a polyunsaturated fatty acid (PUFA) selected from the group consisting of an omega-3 PUFA and an omega-6 PUFA, or a precursor or source thereof, effective to modulate Reelin expression in a tissue or fluid of the patient. In one aspect, the amount of the PUFA is sufficient to increase Reelin expression in a tissue or fluid of the patient.

Yet another embodiment of the present invention relates to a method to prevent, reduce or delay the onset of retinal developmental defects or disorders. This method includes the step of administering to the patient a polyunsaturated fatty acid (PUFA) selected from the group consisting of an omega-3 PUFA and an omega-6 PUFA, or a precursor or source thereof, effective to prevent, reduce or delay the onset of retinal developmental defects or disorders and to compensate for the effects of Reelin deficiency or dysfunction in the patient.

Another embodiment of the present invention relates to a method to prevent, reduce or delay the onset of developmental defects or disorders associated with Reelin deficiency or dysfunction. This method includes the steps of: (a) measuring the expression or biological activity of Reelin in a biological sample from a patient; and (b) administering to the patient a polyunsaturated fatty acid (PUFA) selected from the group consisting of an omega-3 PUFA and an omega-6 PUFA, or a precursor or source thereof, wherein the amount of the PUFA administered is determined based on the measurement of expression or biological activity of the Reelin in the sample. In one aspect, the step of measuring the expression or activity of Reelin further comprises determining the relative expression or activity of individual size forms of Reelin in the sample. In one aspect, the amount of PUFA administered to the patient is determined by comparing the level of expression or biological activity of Reelin in the patient sample to a baseline level of Reelin expression or activity that corresponds to a recommended dosage of the PUFA, and adjusting the dosage of the PUFA for the patient accordingly. In this aspect, the amount of PUFA administered to the patient can be increased relative to the recommended dosage of PUFA when the expression or biological activity of Reelin in the patient is decreased relative to the baseline level. In another aspect, the amount of PUFA administered to the patient is determined by comparing the expression or activity of different Reelin size forms in the patient sample to a baseline profile of Reelin size forms that corresponds to a recommended dosage of PUFA, and adjusting the dosage of the PUFA for the patient accordingly. In this aspect, the amount of PUFA administered to the patient can be increased relative to the recommended dosage of PUFA when the relative expression or activity of one or more Reelin size forms in the patient sample differs from the relative expression or activity of the Reelin size form in the baseline profile. The step of measuring the expression or biological activity of Reelin in a biological sample from the patient can be repeated one or more times subsequent to the administration of the PUFA to the patient, and the amount of PUFA administered to the patient is adjusted according to the repeated measurement of the expression or biological activity of Reelin in the patient. The step of measuring the expression or biological activity of Reelin in a biological sample from the patient can also be repeated intermittently throughout a portion of the life of the patient or throughout the entire life of the patient, and wherein the amount of PUFA administered to the patient is adjusted to correspond to each new measurement of the expression or biological activity of Reelin in the patient. When the expression or biological activity of Reelin in the patient is substantially normal, the PUFA is administered as a supplement to prevent or reduce the risk of development of Reelin deficiency or dysfunction. Patients to be treated using this method include, but are not limited to: a pregnant female, a lactating female, a human adult, a human child or adolescent, a human embryo or fetus, wherein the PUFA is administered to the embryo or fetus by administering the PUFA to the mother of the embryo or fetus, a patient that has or is at risk of developing a neurological disorder or neuropsychiatric disorder associated with Reelin deficiency or dysfunction or a fatty acid binding protein deficiency, a patient that has or is at risk of developing an autoimmune disease associated with Reelin deficiency or dysfunction or a fatty acid binding protein deficiency, or a patient that has or is at risk of developing a developmental defect associated with Reelin deficiency or dysfunction or a fatty acid binding protein deficiency.

Yet another embodiment of the present invention relates to a method to monitor the levels of DHA in the brain of a patient. The method includes the steps of measuring the levels of Reelin expression or biological activity in a biological sample from the patient and estimating the levels of DHA in the brain of the patient based on the measurement of Reelin. In one aspect, the method further includes administering an amount of DHA to the patient corresponding to the measured levels of Reelin expression or biological activity. Preferably, the amount of DHA administered is sufficient to compensate for reduced expression or activity of brain lipid binding proteins in the patient or to improve the activity of brain lipid binding proteins in the patient. The method can also include a step of comparing the level of Reelin expression or biological activity in the biological sample from the patient to a baseline level of Reelin expression or biological activity. The baseline level of Reelin expression or biological activity is correlated with a baseline level of DHA in the brain of a subject, wherein the baseline level is established by a method selected from: (a) establishing a baseline level of Reelin expression or activity from a previous measurement of Reelin expression or activity in a previous sample from the patient, wherein the previous sample was of a same cell type, tissue type or bodily fluid type; or, (b) establishing a baseline level of Reelin expression or activity from control samples of a same cell type, tissue type or bodily fluid type as the sample from the patient, the control samples having been obtained from a population of matched individuals. An estimated low level of DHA in the brain of the patient as compared to the baseline level of DHA can indicate that the patient should be administered an amount of DHA to compensate for the level of DHA in the brain of the patient.

Another embodiment of the present invention relates to a method to diagnose a DHA deficiency in a patient. The method includes the steps of: (a) measuring Reelin expression or biological activity in a biological sample from a patient; (b) comparing the Reelin expression or biological activity in the biological sample to a baseline level of Reelin; and, (c) making a diagnosis of the patient, wherein detection of a difference in the level of Reelin expression or biological activity in the biological sample as compared to the baseline level of Reelin expression or biological activity, indicates a positive diagnosis of DHA deficiency in the patient. In one aspect, detection of a lower level of Reelin expression or biological activity in the biological sample as compared to the baseline level of Reelin expression or biological activity, indicates a positive diagnosis of DHA deficiency in the patient. The biological sample can be chosen from: a cell sample, a tissue sample, and a bodily fluid sample, and is preferably a blood sample. The step (a) of measuring can include measuring Reelin mRNA transcription, such as by reverse transcriptase-PCR (RT-PCR), in situ hybridization, Northern blot, sequence analysis, microarray analysis, or detection of a reporter gene. The step (a) of measuring can include measuring Reelin protein expression, such as by immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting, flow cytometry, or protein microchip or microarray. The step (a) of measuring can include measuring Reelin biological activity, such as by receptor-ligand assay and a phosphorylation assay.

In one aspect of this embodiment, the baseline level is established by a method selected from: (a) establishing a baseline level of Reelin expression or activity in an autologous control sample from the patient, wherein the autologous sample is of a same cell type, tissue type or bodily fluid type as the sample of step (a); (b) establishing a baseline level of Reelin expression or activity that is an average from at least two previous measurements of Reelin expression or activity in a previous sample from the patient, wherein each of the previous samples were of a same cell type, tissue type or bodily fluid type as the sample of step (a), and wherein the previous measurements resulted in a negative diagnosis; or, (c) establishing a baseline level of Reelin expression or activity from control samples of a same cell type, tissue type or bodily fluid type as the sample of step (a), the control samples having been obtained from a population of matched individuals.

Another embodiment of the present invention relates to a method to predict the efficacy of incorporation of HUFA into the phospholipid membranes in a patient. The method includes the steps of: (a) measuring Reelin expression or biological activity in a biological sample from a patient; (b) comparing the Reelin expression or biological activity in the biological sample to a baseline level of Reelin; and (c) predicting the patient efficacy of the incorporation of HUFA into phospholipids membranes, wherein a difference in the level of Reelin expression or biological activity in the biological sample as compared to the baseline level of Reeiin expression or biological activity indicates a modification in the predicted ability of the patient to efficaciously incorporate HUFA into phospholipids membranes. In one aspect, the method includes the additional step of prescribing an amount of DHA to the patient, wherein the amount is determined based on the predicted ability of the patient to efficaciously incorporate HUFA into phospholipids membranes.

Another embodiment of the present invention relates to a method to supplement PUFAs in a female during pregnancy and lactation. The method includes the steps of: (a) measuring the expression or biological activity of Reelin in a biological sample from one or both parents of a fetus or child; and (b) administering a polyunsaturated fatty acid (PUFA) selected from the group consisting of an omega-3 PUFA and an omega-6 PUFA, or a precursor or source thereof to the mother of the fetus or child, wherein the amount of PUFA administered is determined based on the measurement of expression or biological activity of the Reelin in the sample from the parent, wherein the PUFA supplements the PUFA in the female and her fetus or child. Preferably, the PUFA is administered: in an amount sufficient to compensate for reduced expression or activity of brain lipid binding proteins in the fetus or child or to improve the activity of brain lipid binding proteins in the fetus or child. In one aspect, the PUFA is administered in an amount sufficient to decrease the risk of giving birth to an infant, and particularly a male infant, with a Reelin deficiency or dysfunction. In another aspect, the PUFA is administered in an amount sufficient to prevent, delay the onset of, or reduce the symptoms of autism in the mother, child or fetus; in an amount sufficient to prevent, delay the onset of, or reduce the symptoms of neuronal migration disorders in the mother, child or fetus; or in an amount sufficient to prevent, delay the onset of, or reduce the symptoms associated with Reelin deficiency or dysfunction in the mother, child or fetus.

Another embodiment of the present invention relates to a method to supplement PUFAs in a female during pregnancy and lactation to decrease the risk of birth of infants having or at risk of developing a Reelin deficiency or dysfunction. The method includes the steps of: (a) identifying the gender of the fetus carried by a pregnant female; and (b) administering a polyunsaturated fatty acid (PUFA) selected from the group consisting of an omega-3 PUFA and/or an omega-6 PUFA, or a precursor or source thereof to the female during all or a portion of the pregnancy and lactation, to decrease the risk that the fetus will be born with or develop after birth a Reelin deficiency or dysfunction, wherein the administration of the PUFA is increased if the fetus is a male as compared to if the fetus is a female.

Yet another embodiment of the present invention relates to a method to prevent, delay the onset of, or reduce a symptom or disorder associated with Reelin deficiency or dysfunction in a child. The method includes the steps of: (a) measuring the expression and/or biological activity of Reelin in a biological sample from the child; and (b) administering to the child a polyunsaturated fatty acid (PUFA) selected from the group consisting of an omega-3 PUFA and an omega-6 PUFA, or a precursor or source thereof, wherein the amount of PUFA administered is determined based on the measurement of expression or biological activity of the Reelin in the sample. In one aspect, the PUFA is provided in an infant formula supplemented with fatty acids comprising DHA and ARA. In one aspect, the PUFA is administered in an amount sufficient to: compensate for reduced expression or activity of brain lipid binding proteins in the child or to improve the activity of brain lipid binding proteins in the child; prevent, delay the onset of, or reduce the symptoms of autism; or prevent, delay the onset of, or reduce the symptoms of neuronal migration disorders.

Another embodiment of the present invention relates to a method to prevent, delay the onset of, or reduce a symptom of Alzheimer's disease associated with low molecular weight Reelin phenotypes. The method includes the steps of: (a) identifying patients with Reelin deficiency or dysfunction, including patients with low molecular weight Reelin phenotypes; and (b) administering to the patient of (a) a polyunsaturated fatty acid (PUFA) selected from the group consisting of an omega-3 PUFA and an omega-6 PUFA, or a precursor or source thereof sufficient to compensate for the effects of Reelin deficiency or dysfunction in the patient.

Another embodiment of the present invention relates to a method to upregulate fatty acid binding proteins (FABP) in a patient. The method includes the step of administering to a patient a polyunsaturated fatty acid (PUFA) selected from: an omega-3 PUFA and an omega-6 PUFA, or a precursor or source thereof effective to upregulate FABP.

Yet another embodiment of the invention relates to a method to upregulate Reelin expression or activity in a patient, comprising administering to the patient a polyunsaturated fatty acid (PUFA) selected from an omega-3 PUFA and an omega-6 PUFA, or a precursor or source thereof effective to upregulate Reelin expression or activity.

Yet another embodiment of the present invention relates to a method to improve neuronal migration in a patient, comprising administering to the patient a polyunsaturated fatty acid (PUFA) selected from an omega-3 PUFA and an omega-6 PUFA, or a precursor or source thereof effective to improve neuronal migration in the patient. In this aspect, neuronal migration can be measured, for example, by measuring levels of Reelin expression or activity in the patient. Neural function can be measured, for example, by imaging techniques, and phenotypic evaluation.

Another embodiment of the present invention relates to a method to identify neural progenitor cells, comprising detecting Reelin expression or biological activity in a population of cells, wherein a defined level of Reelin expression or biological activity is associated with neural progenitor cells. The method can further include a step of selecting the neural progenitor cells for which Reelin expression or biological activity was detected.

Yet another embodiment of the present invention relates to a method to monitor neural development. The method includes the steps of: (a) providing a population of cells comprising neural progenitor cells; (b) detecting Reelin expression or activity in the population of cells; (c) exposing the population of cells to conditions under which the neural progenitor cells will develop into differentiated neural cells; and (d) monitoring the expression or activity of Reelin in the cells after step (c), to evaluate the development of the neural progenitor cells into differentiated neural cells. The method can further include the step of contacting the population of cells of step (a) with a putative developmental regulatory compound prior to or concurrent with step (b), and determining whether the putative regulatory compound affects the development of the neural progenitor cells into differentiated neural cells by detecting Reelin expression or activity in the population of cells.

Another embodiment of the present invention relates to a method to treat or prevent a disorder associated with a deficiency or dysfunction in fatty acid binding proteins. The method includes the steps of: (a) identifying patients with decreased expression or activity of at least one fatty acid binding protein; and (b) administering to the patient a polyunsaturated fatty acid (PUFA) selected from an omega-3 PUFA and an omega-6 PUFA, or a precursor or source thereof in an amount that is determined be sufficient to compensate for the effects of the decreased expression or activity of the fatty acid binding protein. The fatty acid binding protein is, in one aspect, a brain lipid binding protein (BLBP). The fatty acid binding protein is, in one aspect, a fatty acid binding protein in the heart.

Another embodiment of the present invention is a method to treat or prevent a disorder associated with reduced activity or dysfunction of a receptor for a fatty acid binding protein. The method includes the steps of: (a) identifying patients with reduced activity or dysfunction of a receptor for a fatty acid binding protein; and (b) administering to the patient a polyunsaturated fatty acid (PUFA) selected from an omega-3 PUFA and an omega-6 PUFA, or a precursor or source thereof in an amount that is determined be sufficient to compensate for the effects of the reduced activity or dysfunction of a receptor for a fatty acid binding protein.

Yet another embodiment of the present invention relates to a pharmaceutical composition including an amount of a polyunsaturated fatty acid (PUFA) selected from: an omega-3 PUFA and an omega-6 PUFA, or a precursor or source thereof; and at least one therapeutic compound for treatment or prevention of a disorder associated with Reelin deficiency sufficient to compensate for the reduced expression or activity of fatty acid binding proteins in a patient that has or is at risk of developing a Reelin deficiency. In one aspect, the therapeutic compound is a thyroid medication.

Another embodiment of the present invention relates to a method to diagnose a DHA deficiency in a patient. The method includes the steps of: (a) measuring Reelin expression or biological activity in a biological sample from a patient; (b) comparing the Reelin expression or biological activity in the biological sample to a baseline level of Reelin; (c) measuring thyroid stimulating hormone (TSH) expression and/or biological activity in a biological sample from a patient; (d) comparing the TSH expression or biological activity in the biological sample to a baseline level of TSH; and, (e) making a diagnosis of the patient, wherein detection of a difference in the level of Reelin expression or biological activity in the biological sample as compared to the baseline level of Reelin expression or biological activity, and wherein detection of a difference in the level of TSH expression or biological activity in the biological sample as compared to the baseline level of TSH expression or biological activity, indicates a positive diagnosis of DHA deficiency in the patient. The biological sample can include a cell sample, a tissue sample, and a bodily fluid sample. In one aspect, the patient is pregnant or suspected of being pregnant.

Another embodiment of the present invention relates to a method to supplement PUFAs in a female during pregnancy and lactation. The method includes the steps of: (a) measuring the expression and/or biological activity of Reelin in a biological sample from the mother of a fetus or child; (b) measuring the expression and/or biological activity of thyroid stimulating hormone in the biological sample; (c) administering a polyunsaturated fatty acid (PUFA) selected from the group consisting of an omega-3 PUFA and an omega-6 PUFA, or a precursor or source thereof to the mother of the fetus or child, wherein the amount of PUFA administered is determined based on the measurement of expression or biological activity of the Reelin in the sample from the parent, wherein the PUFA supplements the PUFA in the female and her fetus or child; and (d) administering at least one thyroid medication to the mother of the fetus or child if the measurement of Reelin and thyroid stimulating hormone in the sample from the mother is determined to be low as compared to a baseline level of Reelin and thyroid stimulating hormone.

Yet another embodiment of the present invention relates to a method to diagnose a fetal neurodevelopmental disorder. The method includes the steps of: (a) measuring Reelin expression or biological activity in an amniotic fluid sample from a fetus; (b) comparing the Reelin expression or biological activity in the sample to a baseline level of Reelin; and, (c) making a diagnosis of the fetus, wherein detection of a difference in the level of Reelin expression or biological activity in the sample as compared to the baseline level of Reelin expression or biological activity, indicates a positive diagnosis of a neurodevelopmental disorder in the fetus. In one aspect, a fetus having a positive diagnosis in (c) is administered an amount of Reelin or reelin gene in utero sufficient to treat the neurodevelopmental disorder. In another aspect, a fetus having a positive diagnosis in (c) is administered an amount of Reelin postnatally (e.g., by an infant formula) sufficient to treat the neurodevelopmental disorder.

Yet another embodiment of the present invention relates to a nutritional supplement or oral pharmaceutical, comprising an amount of Reelin sufficient to delay or prevent the development of a Reelin-deficiency or dysfunction or a disease or condition related thereto. In one aspect, the supplement or pharmaceutical is provided in infant formula. In another aspect, the supplement or pharmaceutical is provided to an infant by milk produced by the infant's mother, wherein the mother of the infant is supplemented with Reelin prior to or during lactation.

In any of the above-described methods where a PUFA is administered, the PUFA is, in one aspect, a highly unsaturated fatty acid (HUFA). In another aspect, the PUFA is chosen from: arachidonic acid (ARA), eicosapentaenoic acid (EPA), docosahexaenoic acid (DHA) and docosapentaenoic acid (DPA). In another aspect, the PUFA is chosen from ARA, EPA, and DHA. In yet another aspect, the PUFA is DHA. In another aspect, the source of the PUFA is selected from: fish oil, marine algae, and plant oil. In yet another aspect, when the PUFA is DHA, the precursor of DHA is selected from: α-linolenic acid (LNA), eicosapentaenoic acid (EPA), docosapentaenoic acid (DPA), and blends of precursors selected from the group consisting of LNA, EPA, and DPA. In another aspect, the PUFA is administered in a form selected from: a highly purified algal oil comprising the PUFA in triglyceride form, triglyceride oil comprising the PUFA, phospholipids comprising the PUFA, a combination of protein and phospholipids comprising the PUFA, dried marine microalgae, sphingolipids comprising the PUFA, esters, a free fatty acid, a conjugate of the PUFA with another bioactive molecule, and combinations thereof. A bioactive molecule can include, but is not limited to, a protein, an amino acid, a drug, or a carbohydrate. In one aspect, the PUFA is administered orally.

In another aspect, the PUFA is administered as a formulation comprising the PUFA or precursor or source thereof selected from: chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods or combinations thereof. In this aspect, the PUFA in the formulation may be provided in a form selected from: a highly purified algal oil comprising the PUFA, triglyceride oil comprising the PUFA, phospholipids comprising the PUFA, a combination of protein and phospholipids comprising the PUFA, dried marine microalgae comprising the PUFA, sphingolipids comprising the PUFA, esters of the PUFA, free fatty acid, a conjugate of the PUFA with another bioactive molecule, or combinations thereof. In another aspect, the PUFA is administered in a dosage of from about 0.05 mg of the PUFA per kg body weight of the patient to about 200 mg of the PUFA per kg body weight of the patient. In another aspect, the PUFA can be administered to the patient or subject in combination with one or more additional therapeutic compounds for treating a condition associated with a Reelin deficiency or dysfunction.

### DETAILED DESCRIPTION OF THE INVENTION

The present invention generally relates to a method to use fatty acid supplementation, and particularly, omega-3 and/or omega-6 polyunsaturated fatty acid (PUFA) supplementation (e.g., DHA) to mitigate or compensate for the effect of Reelin deficiency or dysfunction and reduced levels of fatty acid binding proteins in the body, and in one embodiment, in the brain. The method of the invention preferably provides a benefit to a patient in the form of prevention, delay of onset, or the treatment of various diseases and conditions associated with Reelin deficiency or dysfunction and/or reduced fatty acid binding proteins. More specifically, the present invention is directed to the supplementation of patients with PUFAs such as DHA to mitigate or compensate for reduced brain lipid binding proteins and for improper neuronal migration in the brain caused by or associated with low levels, improper expression or dysregulation of the glycoprotein, Reelin. Improper neuron migration has been associated with a variety of neurological disorders including dyslexia, dyspraxia, seizures, epilepsy and attention deficit hyperactivity disorder (ADHD) as well as psychiatric disorders such as schizophrenia, bipolar disorder, depression, Zellweger syndrome, Lissencepahly, Down's Syndrome, Muscle-Eye-Brain Disease, Walker-Warburg Syndrome, Charoct-Marie-Tooth Disease, inclusion body myositis (IBM) and Aniridia.

A proper functioning Reelin signaling pathway is vital to proper neuron migration in the cerebral cortex of the developing brain. Deviations in this pathway can cause an under expression of polyunsaturated fatty acid-specific binding proteins or brain lipid binding proteins (BLBP) in radial glial cells and astrocytes, resulting in shortened radial glial process extensions and thereby improper neuronal migration. Without being bound by theory, the present inventors believe that BLBP is expressed to store and protect polyunsaturated fatty acids, and specifically DHA, from oxidation and phospholipase activity in the developing brain. In the present invention, omega-3 fatty acid supplementation is supplied to patients with Reelin deficiency and/or dysregulation to offset the effects of low BLBP expression by supplying the brain with proper amounts of functional DHA that can be incorporated into phospholipid membranes in the developing glial cells and neurons.

Accordingly, in one embodiment, the present invention generally relates to a method of measuring Reelin as a biomarker, to non-destructively assess or predict DHA levels in the brain and in other, currently inaccessible or difficult-to-access, key components of the central nervous system (CNS). For example, Reelin size forms (Reelin moieties), including Reelin expression and/or biological activity levels can be measured to qualitatively infer the relative amounts of DHA levels in the brain. This measure can be used to indirectly track DHA levels in the brain throughout the entire life of an individual and be used as an indicator for the need of nutritional intervention with DHA at certain points within the life cycle. Prior to the present invention, it was difficult to assess levels of DHA in the brain without potentially harming the patient.

The present invention also relates to a method to prevent, delay the onset of, or treat Reelin deficiency or dysfunction and/or a disease or condition associated with Reelin deficiency or dysfunction, comprising administering to a patient diagnosed with or suspected of having a Reelin deficiency or dysfunction an amount of a PUFA, and particularly an omega-3 PUFA, and more particularly, docosahexaenoic acid (DHA) or a precursor or source thereof, to compensate for the effects of Reelin deficiency or dysfunction in the patient. Prior to the present invention, although DHA had been proposed for use in the treatment of some neurodegenerative disorders, it was not appreciated that there is a specific subset of patients with neurodegenerative disorders for whom the administration of DHA or other PUFA is now predicted to be particularly efficacious. The present invention allows for the identification of such patients via the measurement of Reelin levels in the patient.

The present invention also relates to a method to prevent or reduce developmental defects or disorders associated with Reelin dysfunction or deficiency through the supplemental use of polyunsaturated fatty acids (PUFAs—unsaturated fatty acids having two or more double bonds), and particularly highly unsaturated fatty acids (HUFAs—unsaturated fatty acids having three or more double bonds), and more particularly a HUFA selected from arachidonic acid (ARA), eicosapentaenoic acid (EPA), docosahexaenoic acid (DHA) and docosapentaenoic acid (DPA), and even more particularly omega-3 HUFAs, and more particularly DHA, to: compensate for reduced fatty acid binding protein or function thereof in the patient; compensate for reduced brain lipid binding protein or function thereof in the patient; improve the activity of fatty acid binding proteins in the patient; increase the expression of brain lipid binding proteins (BLBPs) in the patient; improve at least one parameter of the mechanism of action of brain lipid binding proteins in the patient; overcome a deficiency of DHA in central nervous system (CNS) structures and improve the resulting function thereof; increase the incorporation of functional DHA and other PUFAs into the phospholipid membranes of glial cells and neurons in the patient; increase the level of Reelin and/or improve the activity of Reelin in the patient; and/or improve at least one symptom of a disease or condition associated with Reelin deficiency or dysfunction.

Particular embodiments of the invention include, but are not limited to, supplementation with at least one PUFA and/or a precursor or source thereof during pregnancy and/or lactation to prevent disorders associated with Reelin deficiency or dysfunction in children (e.g., autism, neuronal migration disorders); supplementation of adults with low molecular weight Reelin phenotypes to prevent, reduce the onset of, or treat a variety of conditions and diseases, including but not limited to: a neurological disorder or neuropsychiatric disorder, seizures, an autoimmune disorder associated with a neurological dysfunction, or an anti-phospholipid disorder. Such conditions and diseases more particularly include, but are not limited to: schizophrenia, bipolar disorder, dyslexia, dyspraxia, attention deficit hyperactivity disorder (ADHD), epilepsy, autism, Parkinson's Disease, senile dementia, Alzheimer's Disease, peroxisomal proliferator activation disorder (PPAR), multiple sclerosis, diabetes-induced neuropathy, macular degeneration, retinopathy of prematurity, Huntington's Disease, amyotrophic lateral sclerosis (ALS), retinitis pigmentosa, cerebral palsy, muscular dystrophy, cancer, cystic fibrosis, neural tube defects, depression, Zellweger syndrome, Lissencepahly, Down's Syndrome, Muscle-Eye-Brain Disease, Walker-Warburg Syndrome, Charoct-Marie-Tooth Disease, inclusion body myositis (IBM) and Aniridia.

In one embodiment of the invention, PUFA supplementation to a pregnant or lactating female is sufficient to reduce the risk of giving birth to an infant that has or is at risk of developing a Reelin-deficiency or dysfunction. In one aspect, PUFA supplementation is particularly useful for reducing the risk of giving birth to a male infant that has or is at risk of developing a Reelin-deficiency or dysfunction. In one embodiment of the present invention, prior to supplementation of a pregnant female with a PUFA, the gender of the fetus is first determined. The present inventors have found that PUFA supplementation can reduce the risk of birth of an infant with a Reelin deficiency or dysfunction, and in one aspect of the invention, this effect may be particularly efficacious when the fetus is a male. In this embodiment, the pregnant female is supplemented during all or a portion of the pregnancy and/or lactation with a polyunsaturated fatty acid (PUFA) selected from an omega-3 PUFA and/or an omega-6 PUFA, or a precursor or source thereof. If the pregnant female is carrying at least one male fetus, then the PUFA supplementation can be increased as compared to if the pregnant female was carrying a female fetus.

The present invention also relates to a method of measuring Reelin and thyroid stimulating hormone (TSH) to non-destructively assess or predict whether DHA levels in a patient should be supplemented, and particularly during pregnancy. The thyroid is part of a large feedback process. The hypothalamus in the brain releases thyrotropin-releasing hormone (TRH). The release of TRH tells the pituitary gland to release thyroid stimulating hormone (TSH). TSH, circulating in your bloodstream, then causes the thyroid to make thyroid hormones and release them into your bloodstream. TSH can increase the production of Reelin. Therefore, lower than normal TSH levels during pregnancy may be correlated with or contribute to insufficient Reelin levels, which may have a negative impact on the developing fetus. While there are existing tests for TSH (e.g., Abbott Laboratories) in women that are used during pregnancy, to test for a combination of TSH levels and Reelin levels has not been described prior to the present invention. Since TSH can affect several biological functions, the present inventors believe that combined testing of TSH and Reelin levels in a patient will give a more accurate assessment of the risk to the patient (and fetus, in the case of the pregnant woman) for improper neuronal development. Such a dual test is useful, therefore, to assess risks in pregnant women and to provide a PUFA supplementation strategy that is likely to have a positive developmental effect on the fetus. The Reelin levels can be measured as described herein, and at the same time as or before or after levels of thyroid stimulating hormone are measured. Methods for measuring TSH levels in a patient are known in the art and a variety of TSH test kits are commercially available (e.g., Biosafe, Abbott Laboratories). If it is determined that the Reelin and TSH levels are lower than the baseline control level, than DHA or other PUFA supplementation is prescribed for the patient, alone or in combination with thyroid medication. PUFA supplementation has been discussed in detail elsewhere herein. Methods to set and assess Reelin baseline levels are described herein (see below) and are also known in the art (e.g., see PCT Publication No. WO 03/063110). TSH baseline levels for humans are known in the art. For example, a TSH level of between about 0.3-0.5 and about 5.0-6.0 MU/liter or, since 2003 (as most recently revised by the American Association of Clinical Endocrinologists), between about 0.3 and about 3.0 MU/liter, is considered to be a normal (baseline) range for TSH in an individual.

The present invention also relates to a method of modulating Reelin expression in tissues to promote the growth of stem cells through the use of at least one omega-3 and/or omega-6 PUFA and/or a precursor or source thereof.

The present invention also relates to a method to monitor the levels of DHA in the brain of a patient, comprising measuring the levels of Reelin expression and/or biological activity in a biological sample from the patient and estimating the levels of DHA in the brain of the patient based on the measurement of Reelin.

The present inventors have also demonstrated (see Examples section) that one can utilize detection of Reelin concentration in a biological sample from a patient to predict, the DHA content of other tissues, including CNS and reproductive tissue. For example, the Reelin expression and/or biological activity in a patient sample can be measured, obtained or determined as described elsewhere herein. The Reelin levels can be compared to a baseline control, also as described elsewhere herein. Since the present inventors have shown that Reelin deficiency or dysfunction is indicative of a reduced ability to efficaciously incorporate functional HUFA into the body, one can then prescribe an amount of supplemental HUFA (e.g., to be administered as a nutritional or therapeutic composition) that will account for the predicted ability of the patient to incorporate functional HUFA into the body tissues and cells. For example, a patient exhibiting a Reelin deficiency or dysfunction may be prescribed a higher dose of HUFA as compared to a patient who does not have a Reelin deficiency or dysfunction, and similarly, the amount of HUFA indicated for the patient can be adjusted or modified over time according to new evaluations of Reelin expression and/or biological activity in the patient. Therefore, another embodiment of the invention relates to a method to predict the efficacy of incorporation of functional HUFA into the phospholipid membranes in a patient, comprising: (a) measuring Reelin expression or biological activity in a biological sample from a patient; (b) comparing the Reelin expression or biological activity in the biological sample to a baseline level of Reelin; and (c) predicting the patient efficacy of the incorporation of functional HUFA into phospholipids membranes, wherein a difference in the level of Reelin expression or biological activity in the biological sample as compared to the baseline level of Reelin expression or biological activity indicates a modification in the predicted ability of the patient to efficaciously incorporate functional HUFA into phospholipids membranes. In one aspect, the method further includes a step of prescribing an amount of HUFA to the patient, wherein the amount is determined based on the predicted ability of the patient to efficaciously incorporate functional HUFA into phospholipids membranes.

The present invention also relates to a method to improve neuronal migration and/or neural function in a patient, comprising administering to the patient a quantity of at least one omega-3 and/or omega-6 PUFA and/or a precursor or source thereof to improve at least one parameter of neuronal migration and/or neural function in the patient.

The present invention also relates to a method to identify neural progenitor cells, comprising detecting Reelin expression and/or biological activity in a population of cells, wherein a defined level of Reelin expression or biological activity is associated with neural progenitor cells.

The present invention also relates to a method to monitor neural development, comprising: (a) providing a population of cells comprising neural progenitor cells; (b) detecting Reelin expression or activity in the population of cells; (c) exposing the population of cells to conditions under which the neural progenitor cells will develop into differentiated neural cells; and (d) monitoring the expression or activity of Reelin in the cells after step (c), to evaluate the development of the neural progenitor cells into differentiated neural cells.

The present invention also relates to the use of DHA in combination with other polyunsaturated fatty acids (PUFAs) (e.g., EPA, ARA, DPA) in any of the above methods.

The present invention also relates to therapeutic compositions comprising an amount of at least one omega-3 and/or omega-6 PUFA and/or a precursor or source thereof sufficient to compensate for the reduced expression and/or activity of fatty acid binding proteins in a patient that has or is at risk of developing a Reelin deficiency.

The present invention also relates to therapeutic compositions comprising an amount of at least one omega-3 and/or omega-6 PUFA and/or a precursor or source thereof sufficient to compensate for the reduced expression and/or activity of fatty acid binding proteins in a patient that has or is at risk of developing a Reelin deficiency, and at least one therapeutic compound for treatment or prevention of a disorder associated with Reelin deficiency.

The present invention also relates to the use of PUFA supplementation, including DHA, in locations other than the CNS (e.g., associated with heart and/or immune/lymph system) in order to prevent, delay the onset of, or treat deficiencies of fatty acid lipid binding proteins in these locations.

Another embodiment of the present invention relates to a method to diagnose a fetal neurodevelopmental disorder, comprising: (a) measuring Reelin expression or biological activity in an amniotic fluid sample from a fetus; (b) comparing the Reelin expression or biological activity in the sample to a baseline level of Reelin; and, (c) making a diagnosis of the fetus, wherein detection of a difference in the level of Reelin expression or biological activity in the sample as compared to the baseline level of Reelin expression or biological activity, indicates a positive diagnosis of a neurodevelopmental disorder in the fetus. Methods to measure Reelin expression and activity are discussed elsewhere herein. In one aspect, a fetus having a positive diagnosis in (c) is administered an amount of Reelin or reelin gene in utero sufficient to treat the neurodevelopmental disorder. In another embodiment, a fetus having a positive diagnosis in (c) is administered an amount of Reelin postnaturally sufficient to treat the neurodevelopmental disorder. For example, the Reelin can be administered in an infant formula. Amounts of Reelin to be administered to a patient, include from about 1 μg per day to about 10,000 μg per day or more, including any increment in between in 0.1 μg per day increments (e.g., 1 μg per day, 1.1 μg per day, 1.2 μg per day, etc.).

Yet another embodiment of the present invention relates to a nutritional supplement or oral pharmaceutical, comprising an amount of Reelin sufficient to delay or prevent the development of a Reelin-deficiency or dysfunction or a disease or condition related thereto. Such a supplement can be provided in an infant formula or other food product, and in one aspect, is provided to an infant by milk produced by the infant's mother, wherein the mother of the infant is supplemented with Reelin prior to or during lactation.

Various aspects of the invention are described in more detail below.

Reelin is an extracellular signaling glycoprotein (>400 kDa) that is secreted by the Cajal-Retzius cells into the marginal zone of the neocortex of the brain, and although there is evidence that Reelin binds to cadherin-related neuronal receptors and B₁-class integrins, Reelin mainly binds to two members of the low density lipoprotein receptor family, VLDLR and ApoER2, having more affinity to the receptor ApoER2. The binding of Reelin to the extracellular domains of either VLDLR or ApoER2 allows or induces the tyrosine phosphorylation of Dab1, a cytoplasmic adaptor protein in the signaling pathway, by cdk5/p35, a serine/threonine kinase, for example.

Reelin molecules assemble to form a large protein complex, but also may have autocatalytic properties, cleaving the Reelin complex into smaller entities. In the mammalian central nervous system (CNS), Reelin and, in particular, some of its specific size variants (also referred to herein as Reelin size forms or Reelin moieties), have been found to control proper neuronal migration and positioning by inducing the phosphorylation of Dab1 via VLDLR and ApoER2. This neuronal migration is necessary for the normal cortical development of the brain.

The importance of Dab1 tyrosine phosphorylation in Reelin signaling is profound. It may activate, for example, phosphoinositide-3-kinase (PI3K), Akt and Src family kinases (SFKs) (Ballif et al., Molecular Brain Research, 2003, 117, pp 152-159). Due to the activation of these kinases or the upregulation of other proteins downstream in the signaling cascade (Notch, NckB, erbB2, erbB4, neuregulin, including the soluble neuregulin, GGF etc.), astrocytes will morphologically transform by elongation into radial glial cells and upregulate the expression of other neuronal receptors, as well as brain lipid binding proteins (BLBPs) (Brody, T., The Interactive Fly: Gene networks, development, 1996).

The nucleotide sequence encoding Reelin has been cloned in both human and mouse, and the cDNA and encoded amino acid sequences for Reelin, can be found in public databases, such as the National Center for Biotechnology Information (NCBI) database. For example, the nucleotide and amino acid sequences for human or mouse Reelin can be found in the NCBI database under Primary Accession No. U24703 and U79716, respectively (the information in these database Accession Nos. is incorporated herein by reference in its entirety). The amino acid sequences from mouse and human are 94% identical, suggesting that the mouse and human Reelin polypeptides are highly structurally and functionally similar. As discussed in PCT Publication No. WO 03/063110, which is incorporated herein by reference in its entirety, at its N-terminus, Reelin has a cleavable signal peptide followed by a segment similar to F-spondin. Reelin also has eight internal repeats of 350-390 amino acids, each containing an epithelial growth factor-like motif flanked by two related segments. The series of internal repeats is preceded by a hinge domain, and is followed by a highly basic 33 amino acid C-terminal domain.

Reelin is found in nature in one or more different “size forms” (Reelin proteins having different molecular masses), also referred to herein as Reelin moieties”. The molecular mass of full-length Reelin is about 410 kD, and products of natural proteolytic cleavage exist which have molecular masses of, for example, about 330 kD and 180 kD. Any other Reelin size forms that can be detected in an individual are also encompassed by the present invention. These size forms can be readily detected using methods known in the art, including, but not limited to, immunoblotting techniques.

Some embodiments of the present invention include a step of administering to a patient an amount of one or more polyunsaturated fatty acids (PUFAs), and more preferably, highly unsaturated fatty acids (HUFAs), and even more preferably, DHA, or precursors or other sources thereof. Polyunsaturated fatty acids (PUFAs) are critical components of membrane lipids in most eukaryotes (Lauritzen et al., Prog. Lipid Res. 40 1 (2001); McConn et al., Plant J 15, 521 (1998)) and are precursors of certain hormones and signaling molecules (Heller et al., Drugs 55, 487 (1998); Creelman et al., Annu. Rev. Plant Physiol. Plant Mol. Biol. 48, 355 (1997)). According to the present invention, a preferred PUFA is a long chain PUFA, which is defined as a PUFA having eighteen carbons or more.

Any source of PUFA can be used in the compositions and methods of the present invention, including, for example, animal, plant and microbial sources. Preferred polyunsaturated fatty acid (PUFA) sources can be any sources of PUFAs that are suitable for use in the present invention. Preferred polyunsaturated fatty acids sources include biomass sources, such as animal, plant and/or microbial sources. As used herein, the term “lipid” includes phospholipids; free fatty acids; esters of fatty acids; triacylglycerols; diacylglycerides; monoacylglycerides; lysophospholipids; soaps; phosphatides; sterols and sterol esters; carotenoids; xanthophylls (e.g., oxycarotenoids); hydrocarbons; and other lipids known to one of ordinary skill in the art. Examples of animal sources include aquatic animals (e.g., fish, marine mammals, crustaceans, rotifers, etc.) and lipids extracted from animal tissues (e.g., brain, liver, eyes, etc.). Examples of plant sources include macroalgae, flaxseeds, rapeseeds, corn, evening primrose, soy and borage. Examples of microorganisms include algae, protists, bacteria and fungi (including yeast). The use of a microorganism source, such as algae, can provide organoleptic advantages, i.e., fatty acids from a microorganism source may not have the fishy taste and smell that fatty acids from a fish source tend to have. More preferably, the long-chain fatty acid source comprises algae.

Preferably, when microorganisms are the source of long-chain fatty acids, the microorganisms are cultured in a fermentation medium in a fermentor. Alternatively, the microorganisms can be cultured photosynthetically in a photobioreactor or pond. Preferably, the microorganisms are lipid-rich microorganisms, more preferably, the microorganisms are selected from the group consisting of algae, bacteria, fungi and protists, more preferably, the microorganisms are selected from: golden algae, green algae, dinoflagellates, yeast, fungi of the genus Mortierella and Stramenopiles. Preferably, the microorganisms comprise microorganisms of the genus Crypthecodinium and order Thraustochytriales and filamentous fungi of the genus Mortierella, and more preferably, microorganisms are selected from the genus Thraustochytrium, Schizochytrium or mixtures thereof, and more preferably, the microorganisms are selected from the group consisting of microorganisms having the identifying characteristics of ATCC number 20888, ATCC number 20889, ATCC number 20890, ATCC number 20891 and ATCC number 20892, strains of Mortierella schmuckeri and Mortierella alpina, strains of Crypthecodinium cohnii, mutant strains derived from any of the foregoing, and mixtures thereof.

According to the present invention, the terms/phrases “Thraustochytrid”, “Thraustochytriales microorganism” and “microorganism of the order Thraustochytriales” can be used interchangeably and refer to any members of the order Thraustochytriales, which includes both the family Thraustochytriaceae and the family Labyrinthulaceae. The terms “Labyrinthulid” and “Labyrinthulaceae” are used herein to specifically refer to members of the family Labyrinthulaceae. To specifically reference Thraustochytrids that are members of the family Thraustochytriaceae, the term “Thraustochytriaceae” is used herein. Thus, for the present invention, members of the Labyrinthulids are considered to be included in the Thraustochytrids.

Developments have resulted in frequent revision of the taxonomy of the Thraustochytrids. Taxonomic theorists generally place Thraustochytrids with the algae or algae-like protists. However, because of taxonomic uncertainty, it would be best for the purposes of the present invention to consider the strains described in the present invention as Thraustochytrids to include the following organisms: Order: Thraustochytriales; Family: Thraustochytriaceae (Genera: Thraustochytrium, Schizochytrium, Japonochytrium, Aplanochytrium, or Elina) or Labyrinthulaceae (Genera Labyrinthula, Labyrinthuloides, or Labyrinthomyxa). Also, the following genera are sometimes included in either family Thraustochytriaceae or Labyrinthulaceae: Althornia, Corallochytrium, Diplophyrys, and Pyrrhosorus), and for the purposes of this invention are encompassed by reference to a Thraustochytrid or a member of the order Thraustochytriales. It is recognized that at the time of this invention, revision in the taxonomy of Thraustochytrids places the genus Labyrinthuloides in the family of Labyrinthulaceae and confirms the placement of the two families Thraustochytriaceae and Labyrinthulaceae within the Stramenopile lineage. It is noted that the Labyrinthulaceae are sometimes commonly called labyrinthulids or labyrinthula, or labyrinthuloides and the Thraustochytriaceae are commonly called thraustochytrids, although, as discussed above, for the purposes of clarity of this invention, reference to Thraustochytrids encompasses any member of the order Thraustochytriales and/or includes members of both Thraustochytriaceae and Labyrinthulaceae. Recent taxonomic changes are summarized below.

Strains of certain unicellular microorganisms disclosed herein are members of the order Thraustochytriales. Thraustochytrids are marine eukaryotes with an evolving taxonomic history. Problems with the taxonomic placement of the Thraustochytrids have been reviewed by Moss (in “The Biology of Marine Fungi”, Cambridge University Press p. 105 (1986)), Bahnweb and Jackie (ibid. p. 131) and Chamberlain and Moss (BioSystems 21:341 (1988)).

For convenience purposes, the Thraustochytrids were first placed by taxonomists with other colorless zoosporic eukaryotes in the Phycomycetes (algae-like fungi). The name Phycomycetes, however, was eventually dropped from taxonomic status, and the Thraustochytrids were retained in the Oomycetes (the biflagellate zoosporic fungi). It was initially assumed that the Oomycetes were related to the heterokont algae, and eventually a wide range of ultrastructural and biochemical studies, summarized by Barr (Barr. Biosystems 14:359 (1981)) supported this assumption. The Oomycetes were in fact accepted by Leedale (Leedale. Taxon 23:261 (1974)) and other phycologists as part of the heterokont algae. However, as a matter of convenience resulting from their heterotrophic nature, the Oomycetes and Thraustochytrids have been largely studied by mycologists (scientists who study fungi) rather than phycologists (scientists who study algae).

From another taxonomic perspective, evolutionary biologists have developed two general schools of thought as to how eukaryotes evolved. One theory proposes an exogenous origin of membrane-bound organelles through a series of endosymbioses (Margulis, 1970, Origin of Eukarvotic Cells. Yale University Press, New-Haven); e.g., mitochondria were derived from bacterial endosymbionts, chloroplasts from cyanophytes, and flagella from spirochaetes. The other theory suggests a gradual evolution of the membrane-bound organelles from the non-membrane-bounded systems of the prokaryote ancestor via an autogenous process (Cavalier-Smith, 1975, Nature (Lond.) 256:462-468). Both groups of evolutionary biologists however, have removed the Oomycetes and Thraustochytrids from the fungi and place them either with the chromophyte algae in the kingdom Chromophyta (Cavalier-Smith BioSystems 14:461 (1981)) (this kingdom has been more recently expanded to include other protists and members of this kingdom are now called Stramenopiles) or with all algae in the kingdom Protoctista (Margulis and Sagen. Biosystems 18:141 (1985)).

With the development of electron microscopy, studies on the ultrastructure of the zoospores of two genera of Thraustochytrids, Thraustochytrium and Schizochytrium, (Perkins, 1976, pp. 279-312 in “Recent Advances in Aquatic Mycology” (ed. E. B. G. Jones), John Wiley & Sons, New York; Kazama. Can J. Bot. 58:2434 (1980); Barr, 1981, Biosystems 14:359-370) have provided good evidence that the Thraustochytriaceae are only distantly related to the Oomycetes. Additionally, genetic data representing a correspondence analysis (a form of multivariate statistics) of 5-S ribosomal RNA sequences indicate that Thraustochytriales are clearly a unique group of eukaryotes, completely separate from the fungi, and most closely related to the red and brown algae, and to members of the Oomycetes (Mannella et al. Mol. Evol. 24:228 (1987)). Most taxonomists have agreed to remove the Thraustochytrids from the Oomycetes (Bartnicki-Garcia. p. 389 in “Evolutionary Biology of the Fungi” (eds. Rayner, A. D. M., Brasier, C. M. & Moore, D.), Cambridge University Press, Cambridge).

In summary, employing the taxonomic system of Cavalier-Smith (Cavalier-Smith. BioSystems 14:461 (1981); Cavalier-Smith. Microbiol Rev. 57:953 (1993)), the Thraustochytrids are classified with the chromophyte algae in the kingdom Chromophyta (Stramenopiles). This taxonomic placement has been more recently reaffirmed by Cavalier-Smith et al. using the 18s rRNA signatures of the Heterokonta to demonstrate that Thraustochytrids are chromists not Fungi (Cavalier-Smith et al. Phil. Tran. Roy. Soc. London Series BioSciences 346:387 (1994)). This places the Thraustochytrids in a completely different kingdom from the fungi, which are all placed in the kingdom Eufungi.

Currently, there are 71 distinct groups of eukaryotic organisms (Patterson. Am. Nat. 154:S96 (1999)) and within these groups four major lineages have been identified with some confidence: (1) Alveolates, (2) Stramenopiles, (3) a Land Plant-green algae-Rhodophyte Glaucophyte (“plant”) clade and (4) an Opisthokont clade (Fungi and Animals). Formerly these four major lineages would have been labeled Kingdoms but use of the “kingdom” concept is no longer considered useful by some researchers.

As noted by Armstrong, Stramenopile refers to three-parted tubular hairs, and most members of this lineage have flagella bearing such hairs. Motile cells of the Stramenopiles (unicellular organisms, sperm, zoospores) are asymmetrical having two laterally inserted flagella, one long, bearing three-parted tubular hairs that reverse the thrust of the flagellum, and one short and smooth. Formerly, when the group was less broad, the Stramenopiles were called Kingdom Chromista or the heterokont (=different flagella) algae because those groups consisted of the Brown Algae or Phaeophytes, along with the yellow-green Algae, Golden-brown Algae, Eustigmatophytes and Diatoms. Subsequently some heterotrophic, fungal-like organisms, the water molds, and labyrinthulids (slime net amoebas), were found to possess similar motile cells, so a group name referring to photosynthetic pigments or algae became inappropriate. Currently, two of the families within the Stramenopile lineage are the Labyrinthulaceae and the Thraustochytriaceae. Historically, there have been numerous classification strategies for these unique microorganisms and they are often classified under the same order (i.e., Thraustochytriales). Relationships of the members in these groups are still developing. Porter and Leander have developed data based on 18S small subunit ribosomal DNA indicating the thraustochytrid-labyrinthulid clade in monophyletic. However, the dade is supported by two branches; the first contains three species of Thraustochytrium and Ulkenia profunda, and the second includes three species of Labyrinthula, two species of Labyrinthuloides and Schizochytrium aggregatum.

The taxonomic placement of the Thraustochytrids as used in the present invention is therefore summarized below:

### Kingdom: Chromophyta (Stramenopiles)

### Phylum: Heterokonta

### Order: Thraustochytriales (Thraustochytrids)

### Family: Thraustochytriaceae or Labyrinthulaceae

### Genera: Thraustochytrium, Schizochytrium, Japonochytrium, Aplanochytrium, Elina, Labyrinthula, Labyrinthuloides, or Labyrinthulomyxa

Some early taxonomists separated a few original members of the genus Thraustochytrium (those with an amoeboid life stage) into a separate genus called Ulkenia. However it is now known that most, if not all, Thraustochytrids (including Thraustochytrium and Schizochytrium), exhibit amoeboid stages and as such, Ulkenia is not considered by some to be a valid genus. As used herein, the genus Thraustochytrium will include Ulkenia.

Despite the uncertainty of taxonomic placement within higher classifications of Phylum and Kingdom, the Thraustochytrids remain a distinctive and characteristic grouping whose members remain classifiable within the order Thraustochytriales. Information regarding such microorganisms and methods of culturing such microorganisms can be found in U.S. Pat. Nos. 5,407,957; 5,130,242 and 5,340,594, which are incorporated herein by reference in their entirety.

Lipids covered by the present invention include lipids comprising a polyunsaturated fatty acid, more particularly, a long chain polyunsaturated fatty acid, and even more particularly, a polyunsaturated fatty acid present in said lipid having a carbon chain length of at least 18, 20 or 22. Such polyunsaturated fatty acid can have at least 3 or at least 4 double bonds. More particularly, the polyunsaturated fatty acid can include docosahexaenoic acid (at least 10, 20, 30 or 35 weight percent), docosapentaenoic acid (at least 5, 10, 15, or 20 weight percent), and/or arachidonic acid (at least 20, 30, 40 or 50 weight percent). Polyunsaturated fatty acids include free fatty acids and compounds comprising PUFA residues, including phospholipids; esters of fatty acids; triacylglycerols; diacylglycerides; monoacylglycerides; lysophospholipids; phosphatides; etc.

Sources of phospholipids include poultry eggs, enriched poultry eggs, algae, fish, fish eggs, and genetically engineered (GE) plant seeds or algae.

Particularly preferred sources of PUFAs, including DHA include, but are not limited to, fish oil, marine algae, and plant oil.

Preferred precursors of the PUFA, DHA, include, but are not limited to, α-linolenic acid (LNA); eicosapentaenoic acid (EPA); docosapentaenoic acid (DPA); blends of LNA, EPA, and/or DPA.

In one embodiment of the invention, blends of fatty acids and particularly, omega-3 fatty acids and omega-6 fatty acids can be used in the methods of the invention. Preferred PUFAs include omega-3 and omega-6 polyunsaturated fatty acids with three or more double bonds. Omega-3 PUFAs are polyethylenic fatty acids in which the ultimate ethylenic bond is three carbons from and including the terminal methyl group of the fatty acid and include, for example, docosahexaenoic acid C22:6(n-3) (DHA) and omega-3 docosapentaenoic acid C22:5(n-3) (DPAn-3). Omega-6 PUFAs are polyethylenic fatty acids in which the ultimate ethylenic bond is six carbons from and including the terminal methyl group of the fatty acid and include, for example, arachidonic acid C20:4(n-6) (ARA), C22:4(n-6), omega-6 docosapentaenoic acid C22:5(n-6) (DPAn-6) and dihomogammalinolenic acid C20:3(n-6)(dihomo GLA).

In accordance with the present invention, the long-chain fatty acids that are used in the supplements and therapeutic compositions described herein are in a variety of forms. For example, such forms include, but are not limited to: a highly purified algal oil comprising the PUFA, triglyceride oil comprising the PUFA, phospholipids comprising the PUFA, a combination of protein and phospholipids comprising the PUFA, dried marine microalgae comprising the PUFA, sphingolipids comprising the PUFA, esters of the PUFA, free fatty acid, a conjugate of the PUFA with another bioactive molecule, and combinations thereof. Bioactive molecules can include any suitable molecule, including, but not limited to, a protein, an amino acid (e.g. naturally occurring amino acids such as DHA-glycine, DHA-lysine, or amino acid analogs), a drug, and a carbohydrate.

The forms outlined herein allow flexibility in the formulation of foods with high sensory quality, dietary supplements, and pharmaceutical agents. For example, currently available microalgal oils contain about 40% DHA. These oils can be turned into ester form and then purified using techniques such as molecular distillation to extend the DHA content to 70% and greater, providing a concentrated product that can be useful in products with size constraints, i.e. small serving sizes such as infant foods or dietary supplements with limited feasible pill size. Use of oil and phospholipid combinations helps to enhance the oxidative stability and therefore sensory and nutritional quality of microalgal oil. Oxidative breakdown compromises the nutritional and sensory quality of PUFAs in triglyceride form. By employing the phospholipid form, the desired PUFAs are more stable and the fatty acids are more bioavailable then when in the triglyceride form. Although microbial oils are more stable than typical fish oils, both are subject to oxidative degradation. Oxidative degradation decreases the nutritional value of these fatty acids. Additionally, oxidized fatty acids are believed to be detrimental to good health. The use of phospholipid DHA/DPA/ARA/dihomo-GLA, a more stable fatty acid system, enhances the health and nutritional value of these supplements. Phospholipids are also easier to blend into aqueous systems than are triglyceride oils. Use of protein and phospholipid combinations allows for the formulation of more nutritionally complex foods as both protein and fatty acids are provided. Use of dried marine microalgae provides high temperature stability for the oil within it and is advantageous for the formulation of foods baked at high temperature.

In one embodiment of the invention, a source of the desired phospholipids includes purified phospholipids from eggs, plant oils, and animal organs prepared via the Friolex process and phospholipid extraction process (PEP) (or related processes) for the preparation of nutritional supplements rich in DHA, DPA, ARA and/or dihomo-GLA. The Friolex and PEP, and related processes are described in greater detail in PCT Patent Nos. PCT/IB01/00841, entitled “Method for the Fractionation of Oil and Polar Lipid-Containing Native Raw Materials”, filed Apr. 12, 2001, published as WO 01/76715 on Oct. 18, 2001; PCT/IB01/00963, entitled “Method for the Fractionation of Oil and Polar Lipid-Containing Native Raw Materials Using Alcohol and Centrifugation”, filed Apr. 12, 2001, published as WO 01/76385 on Oct. 18, 2001; and PCT/DE95/01065 entitled “Process For Extracting Native Products Which Are Not Water-Soluble From Native Substance Mixtures By Centrifugal Force”, filed Aug. 12, 1995, published as WO 96/05278 on Feb. 22, 1996; each of which is incorporated herein by reference in its entirety.

Preferably, the highly purified algal oil comprising: the desired PUFA in triglyceride form, triglyceride oil combined with phospholipid, phospholipid alone, protein and phospholipid combination, or dried marine microalgae, comprise fatty acid residues selected from the group made up of DHA and/or DPA(n-3) and/or DPA(n-6) and/or ARA and/or dihomo-GLA. More preferably, the highly purified algal oil comprising the desired PUFA in triglyceride form, triglyceride oil combined with phospholipid, phospholipid alone, protein and phospholipid combination, or dried marine microalgae, comprise fatty acid residues selected from the group made up of DHA, ARA or DPA(n-6). More preferably, the highly purified algal oil comprising the desired PUFA in triglyceride form, triglyceride oil combined with phospholipid, phospholipid alone, protein and phospholipid combination, or dried marine microalgae, comprise fatty acid residues selected from the group made up of DHA and DPA(n-6). In a most preferred embodiment, the highly purified algal oil comprising the desired PUFA in triglyceride form, triglyceride oil combined with phospholipid, phospholipid alone, protein and phospholipid combination, or dried marine microalgae, comprise fatty acid residues of DHA.

Although fatty acids such as DHA can be administered topically or as an injectable, the most preferred route of administration is oral administration. Preferably, the fatty acids (e.g., PUFAs) are administered to patients in the form of nutritional supplements and/or foods and/or pharmaceutical formulations and/or beverages, more preferably foods, beverages, and/or nutritional supplements, more preferably, foods and beverages, more preferably foods.

For infants, the fatty acids are administered to infants as infant formula, weaning foods, jarred baby foods, and infant cereals.

Any biologically acceptable dosage forms, and combinations thereof, are contemplated by the inventive subject matter. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods and combinations thereof. The preparations of the above dosage forms are well known to persons of ordinary skill in the art. Preferably, a food that is enriched with the desired PUFA is selected from the group including, but not limited to: baked goods and mixes; chewing gum; breakfast cereals; cheese products; nuts and nut-based products; gelatins, pudding, and fillings; frozen dairy products; milk products; dairy product analogs; soft candy; soups and soup mixes; snack foods; processed fruit juice; processed vegetable juice; fats and oils; fish products; plant protein products; poultry products; and meat products.

The amount of a PUFA to be administered to a patient can be any amount suitable to provide the desired result of: compensation for reduced fatty acid binding protein or function thereof in the patient; compensation for reduced brain lipid binding protein or function thereof in the patient; improve the activity of fatty acid binding proteins in the patient; increase the expression of brain lipid binding proteins (BLBPs) in the patient; improve at least one parameter of the mechanism of action of brain lipid binding proteins in the patient; overcome a deficiency of fatty acids such as DHA in central nervous system (CNS) structures and the resulting function thereof; increase the incorporation of functional fatty acids such as DHA into the phospholipid membranes of glial cells and neurons in the patient; increase the level of Reelin and/or improve the activity of Reelin in the patient; and/or improve at least one symptom of a disease or condition associated with Reelin deficiency or dysfunction. In one embodiment, a fatty acid (PUFA) is administered in a dosage of from about 0.05 mg of the PUFA per kg body weight of the patient to about 200 mg of the PUFA per kg body weight of the patient or higher, including any increment in between, in 0.01 mg increments (e.g., 0.06 mg, 0.07 mg, etc.), or in amounts ranging between about 50 mg and about 20,000 mg per subject per day via oral, injection, emulsion or total parenteral nutrition, topical, intraperitoneal, placental, transdermal, or intracranial delivery. A typical capsule DHA supplement for example, can be produced in 100 mg to 200 mg doses per capsule, although the invention is not limited to capsule forms or capsules containing these amounts of DHA or another PUFA. In one embodiment of the invention, the PUFA supplement is administered to the patient in combination with one or more additional therapeutic compounds for treating a condition associated with a Reelin deficiency or dysfunction. Such therapeutic compounds will be well known to those of skill in the art for the particular disease or condition being treated.

As discussed above, administration of a PUFA supplement such as DHA to the selected patient preferably provides one or more of the following results: compensates for reduced fatty acid binding protein or function thereof in the patient; compensates for reduced brain lipid binding protein or function thereof in the patient; improves the activity of fatty acid binding proteins in the patient; improves at least one parameter of the mechanism of action of brain lipid binding proteins in the patient; results in increased incorporation of functional DHA into the phospholipid membranes of glial cells and neurons in the patient; increases the level of Reelin and/or improves the activity of Reelin in the patient. In one embodiment, the patient suffers from a disease or condition associated with the Reelin deficiency or dysfunction, and administration of the PUFA to the patient improves at least one symptom of the disease or condition.

A patient to be treated can be at risk of developing or may already suffer from any disease or condition associated with the Reelin deficiency or dysfunction. Such diseases and conditions, include, but are not limited to: neurological disorder or neuropsychiatric disorder, seizures, autoimmune disorders associated with a neurological dysfunction, and an anti-phospholipid disorder. More specifically, such diseases or conditions include, but are not limited to: schizophrenia, bipolar disorder, dyslexia, dyspraxia, attention deficit hyperactivity disorder (ADHD), epilepsy, autism, Parkinson's Disease, senile dementia, Alzheimer's Disease, peroxisomal proliferator activation disorder (PPAR), multiple sclerosis, diabetes-induced neuropathy, macular degeneration, retinopathy of prematurity, Huntington's Disease, amyotrophic lateral sclerosis (ALS), retinitis pigmentosa, cerebral palsy, muscular dystrophy, cancer, cystic fibrosis, neural tube defects, depression, Zellweger syndrome, Lissencepahly, Down's Syndrome, Muscle-Eye-Brain Disease, Walker-Warburg Syndrome, Charoct-Marie-Tooth Disease, inclusion body myositis (IBM) and Aniridia.

Preferably, administration of a PUFA such as DHA to the patient prevents, delays the onset of, or reduces the severity or duration of at least one symptom of the disease or condition associated with Reelin deficiency or dysfunction. In a preferred embodiment, the patient no longer suffers discomfort and/or altered function resulting from or associated with the inappropriate Reelin levels or function as a result of the methods of the invention.

As such, a therapeutic benefit is not necessarily a cure for a particular disease or condition, but rather, preferably encompasses a result which most typically includes alleviation of the disease or condition, elimination of the disease or condition, reduction of a symptom associated with the disease or condition, compensation for or restoration to normal of a cellular or intracellular mechanism, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, a “patient” does not necessarily have or is not necessarily at risk of developing a disease, condition or Reelin deficiency or dysfunction, but rather, the term can be used interchangeably with “subject”, “individual”, and most generally refers to an individual animal (e.g., a human subject or domesticated animal) who is to be evaluated, diagnosed, treated or otherwise impacted by a method or composition of the invention.

One step of many of the above-identified methods of the present invention described herein includes detecting, measuring or evaluating Reelin expression or biological activity in a biological sample from a patient. The sample can be a cell sample, a tissue sample and/or a bodily fluid sample. According to the present invention, the term “cell sample” can be used generally to refer to a sample of any type which contains cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells which are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body, which typically includes several cell types and/or cytoskeletal structure, which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, may contain cells and is a fluid obtained by any method suitable for the particular bodily fluid to be sampled. Bodily fluids suitable for sampling include, but are not limited to, blood, mucous, seminal fluid, saliva, breast milk, bile and urine. In a preferred embodiment of the invention, the biological sample is a blood sample, including any blood fraction (e.g., whole blood, plasma, serum).

In general, the sample type (i.e., cell, tissue or bodily fluid) is selected based on the accessibility of the sample and purpose of the method. Typically, biological samples that can be obtained by the least invasive method are preferred (e.g., blood), although in some embodiments, it may be useful or necessary to obtain a cell or tissue sample for evaluation. Once a sample is obtained from the patient, the sample is evaluated for detection of Reelin expression or biological activity in the cells of the sample. The phrase “Reelin expression” can generally refer to Reelin mRNA transcription or Reelin protein translation (e.g., detecting the amount of Reelin protein in a sample). Preferably, the method of detecting Reelin expression or biological activity in the patient is the same or qualitatively equivalent to the method used for detection of Reelin expression or biological activity in the sample used to establish the baseline or control level of Reelin.

Methods suitable for detecting Reelin transcription include any suitable method for detecting and/or measuring mRNA levels from a fluid, cell or cell extract. Such methods include, but are not limited to: polymerase chain reaction (PCR), reverse transcriptase PCR (RT-PCR), in situ hybridization, Northern blot, sequence analysis, microarray analysis, and detection of a reporter gene. Such methods for detection of transcription levels are well known in the art, and many of such methods are described, for example, in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid. and Glick et al., ibid. are incorporated by reference herein in their entireties. Measurement of Reelin transcription is primarily suitable when the sample is a cell or tissue sample; therefore, when the sample is a bodily fluid sample containing cells or cellular extracts, the cells are typically isolated from the bodily fluid to perform the expression assay.

Reelin expression can also be identified by detection of Reelin translation (i.e., detection of Reelin protein in the sample). Methods suitable for the detection of Reelin protein include any suitable method for detecting and/or measuring proteins from a fluid, cell or cell extract. Such methods include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and protein microchip or microarray. Such methods are well known in the art. Antibodies against Reelin have been produced and described in the art (e.g., see Ogawa et al., 1995, Neuron, 14:890-912; DeBergeyck et al., 1998, J; Neurosci. 15 Meth., 82: 17-24) and can be used in many of the assays for detection of Reelin protein. In PCT Publication No. WO 03/063110, for example, immunoblotting techniques are used to detect the quantity of Reelin size forms in blood samples from patients with various neurological/psychological conditions and compare to Reelin levels in a baseline control population. Such methods are useful for detecting Reelin in a biological sample, although it will be apparent to those of skill in the art that a variety of Reelin detection and measurement techniques can be used to evaluate the Reelin status of an individual.

Alternatively, one can readily produce antibodies against Reelin using techniques well known in the art. Antibodies that selectively bind to Reelin in the sample can be produced using Reelin protein information available in the art. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.). Antibodies useful in the assay kit and methods of the present invention can include polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

As discussed above, Reelin is found in patients in one or more different “size forms” (Reelin proteins having different molecular weights). These “Reelin moieties” or “size forms” can also be detected and compared one to another, or a particular size form of Reelin (Reelin moiety) can be compared to the same Reelin moiety (a Reelin moiety of the same molecular weight) in a baseline or control sample. In addition, one can detect the ratio, or profile, of different Reelin size forms in a biological sample from a patient, and compare the profile to that from a baseline control. Particularly useful Reelin size forms (moieties) to detect include those having apparent molecular masses of about 410 kD (full length Reelin) and naturally occurring proteolytic cleavage products of about 330 kD, and 180 kD. Reelin size forms can be detected and distinguished from one another using many of the above-identified methods for detection of Reelin protein. Methods of detecting the level of Reelin protein in a sample, including Reelin size forms, have also been described in detail in PCT Publication WO 03/063110, which is incorporated herein by reference in its entirety. For example, in this publication, it was determined that the ratio and quantities of Reelin size forms in patients with major depression, schizophrenia, bipolar disorder were statistically significantly different than the levels of the Reelin size forms in normal (non-affected) controls. Similar results were found in autistic patients and their family members as compared to control subjects without autism in the family. Therefore, the detection of changes in relative levels of Reelin size forms, as well as overall levels of Reelin, in a biological sample of a test subject, can readily be compared to control or baseline levels to evaluate the Reelin status in a given test subject and thereby identify Reelin deficiencies or dysfunctions, including Reelin abnormalities.

The term, “Reelin biological activity” refers to any biological action of the Reelin protein, including, but not limited to, binding to a Reelin receptor (e.g., cadherin-related neuronal receptors, B₁-class integrins, low density lipoprotein receptors, and particularly, VLDLR and ApoER2), activation of a Reelin receptor, activation of Reelin cell signal transduction pathways (e.g., the tyrosine phosphorylation of Dab1 by cdk5/p35); and downstream biological events that occur as a result of Reelin binding to a receptor (e.g., activation of phosphoinositide-3-kinase (PI3K), Akt and Src family kinases (SFKs); upregulation of proteins such as Notch, NckB, erbB2, erbB4, neuregulin; morphological transformation of astrocytes into radial glial cells; upregulation of the expression of neuronal receptors; upregulation of brain lipid binding proteins (BLBPs); etc.). Methods to detect Reelin biological activity are known in the art and include, but are not limited to, receptor-ligand assays, and phosphorylation assays.

The diagnostic and monitoring methods of the present invention have several different uses. First, the method can be used to diagnose and monitor a subset of patients who have Reelin deficiency or dysfunction within a larger pool of patients having a given condition (e.g., a neurological condition), who are most likely to be benefited by the methods of the present invention (e.g., by supplementation with PUFAs). The method can also be used to diagnose and monitor patients by identifying patients that have DHA or other PUFA deficiency, or a deficiency or dysfunction in fatty acid binding proteins (FABP), or the potential for DHA or other PUFA deficiency or a FABP deficiency or dysfunction, in a patient. The patient can be an individual who is suspected of having a DHA or other PUFA deficiency or a FABP deficiency or dysfunction, or an individual who is presumed to be healthy, but who is undergoing a routine screening for DHA or other PUFA deficiency or a FABP deficiency or dysfunction. The patient can also be an individual who has previously been diagnosed with DHA or other PUFA deficiency or a FABP deficiency or dysfunction and treated, and who is now under routine surveillance for recurring DHA or other PUFA deficiency or a FABP deficiency or dysfunction.

The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, or a need for PUFA supplementation, for example, has been identified. In contrast, a “negative diagnosis” indicates that the disease or condition, or a potential for developing the disease or condition, or a need for PUFA supplementation, has not been identified. Therefore, in the present invention, a positive diagnosis (i.e., a positive assessment) of DHA or other PUFA deficiency or a FABP deficiency or dysfunction, or the potential therefor, means that the indicators (e.g., signs, symptoms) of DHA or other PUFA deficiency or a FABP deficiency or dysfunction according to the present invention (e.g., Reelin deficiency or dysfunction) have been identified in the sample obtained from the patient. Such a patient can then be prescribed treatment to reduce or eliminate the DHA or other PUFA deficiency or a FABP deficiency or dysfunction. Similarly, a negative diagnosis (i.e., a negative assessment) for DHA or other PUFA deficiency or a FABP deficiency or dysfunction, or a potential therefor, means that the indicators of DHA or other PUFA deficiency or a FABP deficiency or dysfunction, or a likelihood of developing DHA or other PUFA deficiency or a FABP deficiency or dysfunction as described herein (e.g., Reelin deficiency or dysfunction), have not been identified in the sample obtained from the patient. In this instance, the patient is typically not prescribed any treatment, or may be placed on low level DHA or other PUFA supplementation, but may be reevaluated at one or more time points in the future to again assess DHA or other PUFA deficiency or a FABP deficiency or dysfunction. Baseline levels for this particular embodiment of the method of assessment of the present invention are typically based on a “normal” or “healthy” sample from the same bodily source as the test sample (i.e., the same tissue, cells or bodily fluid), as discussed in detail below.

In one embodiment of this method of the present invention, the method is used to monitor the success, or lack thereof, of a treatment for Reelin deficiency or dysfunction, PUFA deficiency, fatty acid binding protein deficiency or dysfunction, or a condition or disease related thereto in a patient that has been diagnosed as having one of the above conditions. In this embodiment, a baseline level of Reelin expression or biological activity typically includes the previous level of Reelin expression or biological activity detected in a sample from the patient to be monitored, so that a new level of Reelin expression or biological activity can be compared to determine whether Reelin, PUFA and/or fatty acid binding protein expression or function is decreasing, increasing, or substantially unchanged as compared to the previous, or first sample. In addition, or alternatively, a baseline established as a “normal” or “healthy” level of Reelin expression or biological activity can be used in this embodiment. This embodiment allows the physician or care provider to monitor the success, or lack of success, of a treatment (e.g., PUFA supplementation) that the patient is receiving for a given condition (e.g. a neurological disorder), and can help the physician to determine whether the treatment should be modified (e.g., whether PUFA supplementation should be increased, decreased, or remain substantially the same). In one embodiment of the present invention, the method includes additional steps of modifying PUFA supplementation treatment for the patient based on whether an increase or decrease in PUFA deficiency is indicated by evaluation of Reelin expression and/or biological activity in the patient.

Accordingly, the diagnostic and monitoring methods of the present invention include a step of comparing the level of Reelin expression or biological activity detected in a patient sample to a baseline level of Reelin expression or biological activity. According to the present invention, a “baseline level” is a control level, and in some embodiments, a normal level, of Reelin expression or activity against which a test level of Reelin expression or biological activity (i.e., in the patient sample) can be compared. Therefore, it can be determined, based on the control or baseline level of Reelin expression or biological activity, whether a sample to be evaluated has a measurable increase, decrease, or substantially no change in Reelin expression or biological activity, as compared to the baseline level. As discussed herein, the baseline level can be indicative of the levels and/or function of fatty acid binding proteins in the patient and particularly, of the levels of PUFA (e.g., DHA) in the patient, and can be used to establish a protocol for DHA and/or other PUFA supplementation in the patient. For example, the baseline level of Reelin can be indicative of the DHA level or other PUFA level in the brain or other tissue expected in a normal (i.e., healthy or negative control) patient. Therefore, the term “negative control” used in reference to a baseline level of Reelin expression or biological activity refers to a baseline level established in a sample from the patient or from a population of individuals, which is believed to be normal with regard to Reelin expression and/or function. In another embodiment, a baseline can be indicative of a positive diagnosis of DHA deficiency or of fatty acid binding protein deficiency or dysfunction. Such a baseline level, also referred to herein as a “positive control” baseline, refers to a level of Reelin expression or biological activity established in a sample from the patient, another patient, or a population of individuals, wherein the Reelin level or function in the sample was believed to correspond to a deficiency in DHA or other PUFA or a fatty acid binding protein or to a disease or condition associated with Reelin deficiency or dysfunction. In yet another embodiment, the baseline level can be established from a previous sample from the patient being tested, so that Reelin status and PUFA status of a patient can be monitored over time. Methods for detecting Reelin expression or biological activity are described in detail above. The method for establishing a baseline level of Reelin expression or activity is selected based on the sample type, the tissue or organ from which the sample is obtained, the status of the patient to be evaluated, and, as discussed above, the focus or goal of the assay (e.g., initial diagnosis, monitoring). Preferably, the method is the same method that will be used to evaluate the sample in the patient.

In one embodiment, the baseline level of Reelin expression or biological activity is established in an autologous control sample obtained from the patient. The autologous control sample can be a sample of isolated cells, a tissue sample or a bodily fluid sample, and is preferably a bodily fluid sample. According to the present invention, and as used in the art, the term “autologous” means that the sample is obtained from the same patient from which the sample to be evaluated is obtained. Preferably, the control sample is obtained from the same fluid, organ or tissue as the sample to be evaluated, such that the control sample serves as the best possible baseline for the sample to be evaluated. This embodiment is most often used when a previous reading from the patient has been established as either a positive or negative diagnosis of Reelin deficiency or dysfunction or DHA deficiency. This baseline can then be used to monitor the ongoing progression of the patient toward or away from a disease or condition, or to monitor the success of therapy (e.g., PUFA supplementation). In this embodiment, a new sample is evaluated periodically (e.g., at annual physicals), and the preventative or therapeutic treatment via fatty acid supplementation is determined at each point. For the first evaluation, an alternate control can be used, as described below, or additional testing may be performed to confirm an initial negative or positive diagnosis of Reelin deficiency or dysfunction, if desired, and the value for Reelin expression or biological activity from the patient sample can be used as a baseline thereafter. This type of baseline control is frequently used in other clinical diagnosis procedures where a “normal” level may differ from patient to patient and/or where obtaining an autologous control sample at the time of diagnosis is either not possible, not practical or not beneficial.

Another method for establishing a baseline level of Reelin expression or biological activity is to establish a baseline level of Reelin expression or biological activity from control samples, and preferably control samples that were obtained from a population of matched individuals. It is preferred that the control samples are of the same sample type as the sample type to be evaluated for Reelin expression or biological activity. According to the present invention, the phrase “matched individuals” refers to a matching of the control individuals on the basis of one or more characteristics which are suitable for the parameter type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). For example, levels of Reelin expression in the blood of a normal individual may be higher in individuals of a given classification (e.g., elderly versus teenagers, women versus men). To establish a control or baseline level of Reelin expression or biological activity, samples from a number of matched individuals are obtained and evaluated for Reelin expression or biological activity. The sample type is preferably of the same sample type and obtained from the same organ, tissue or bodily fluid as the sample type to be evaluated in the test patient. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed to establish a suitable baseline level using methods standard in the art for establishing such values.

A baseline, such as that described above, can be a negative control baseline, such as a baseline established from a population of apparently normal control individuals. Alternatively, as discussed above, such a baseline can be established from a population of individuals that have been positively diagnosed as having Reelin deficiency or dysfunction so that one or more baseline levels can be established for use in evaluating a patient. The level of Reelin expression or biological activity in the patient sample is then compared to each of the baseline levels to determine to which type of baseline (positive or negative) the Reelin level of the patient is statistically closest. It will be appreciated that a given patient sample may fall between baseline levels such that the best diagnosis is that the patient is perhaps beginning to show a Reelin deficiency or dysfunction indicative of the need for at least some fatty acid supplementation, and is perhaps in the process of advancing to the higher stage. The goal of the invention is to reverse, correct, or compensate for such advancing disease.

It will be appreciated by those of skill in the art that a baseline need not be established for each assay as the assay is performed but rather, a baseline can be established by referring to a form of stored information regarding a previously determined baseline level of Reelin expression for a given control sample, such as a baseline level established by any of the above-described methods. Such a form of stored information can include, for example, but is not limited to, a reference chart, listing or electronic file of population or individual data regarding “normal” (negative control) or positive Reelin expression; a medical chart for the patient recording data from previous evaluations; or any other source of data regarding baseline Reelin expression that is useful for the patient to be diagnosed.

After the level of Reelin expression or biological activity is detected in the sample to be evaluated, such level is compared to the established baseline level of Reelin expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in Reelin expression or biological activity over the baseline level, or whether there is no statistically significant difference between the test and baseline levels. After comparing the levels of Reelin expression or biological activity in the samples, the final step of making a diagnosis, monitoring, or determining treatment of the patient can be performed.

Detection of a decreased level of Reelin expression or biological activity (or at least of some size forms of Reelin) in the sample to be evaluated (i.e., the test sample) as compared to the baseline level generally indicates that, as compared to the baseline sample, the patient will have decreased FABP levels and decreased DHA or other PUFA incorporation into the brain tissue. More specifically, if the baseline is a normal or negative control sample, a detection of decreased Reelin expression or biological activity in the test sample as compared to the control sample indicates that the patient has decreased and likely inappropriate DHA or other PUFA levels (a DHA or other PUFA deficiency). If the baseline sample is a previous sample from the patient (or a population control) and is representative of a positive diagnosis of Reelin deficiency or dysfunction in the patient, a detection of decreased Reelin expression or biological activity in the sample as compared to the baseline indicates that the patient condition is worsening, rather than improving and that treatment should be reevaluated or adjusted.

Detection of an increased level of Reelin expression or biological activity (or at least of some Reelin size forms) in the sample to be evaluated (i.e., the test sample) as compared to the baseline level indicates that, as compared to the baseline sample, the patient is experiencing less FABP expression or function, and less DHA or other PUFA deficiency. More specifically, if the baseline is a normal or negative control, a detection of increased Reelin expression or biological activity in the test sample as compared to the control sample indicates that the test sample is most likely also normal and perhaps that the patient produces and/or consumes more DHA or other PUFAs than the average normal patient. If the baseline sample is a previous sample from the patient (or from a population control) and is representative of a positive diagnosis of Reelin deficiency or dysfunction in the patient (i.e., a positive control), a detection of increased Reelin expression or biological activity in the sample as compared to the baseline indicates that the test sample is predictive of an improved level or function of FABP and of increased DHA or other PUFAs in the brain of the patient.

Finally, detection of Reelin expression that is not statistically significantly different than the Reelin expression or biological activity in the baseline sample indicates that, as compared to the baseline sample, no difference in FABP status or DHA (or other PUFA) status is indicated in the test sample. More specifically, if the baseline is a normal or negative control, a detection of Reelin expression or biological activity in the test sample that is not statistically significantly different than the baseline sample indicates that the test sample is essentially normal and is not currently indicative of an FABP or DHA or other PUFA deficiency or disease or condition related to Reelin deficiency or dysfunction. If the baseline sample is a previous sample from the patient (or from a population control) and is representative of a positive diagnosis of Reelin deficiency or dysfunction in the patient (i.e., a positive control), a detection of Reelin expression or biological activity in the sample that is not statistically significantly different than the baseline indicates that the patient has a substantially similar Reelin deficiency or dysfunction and should be treated accordingly. Such a diagnosis might suggest to a clinician that a treatment currently being prescribed, for example, is ineffective in controlling the condition.

In order to establish a diagnosis of a change as compared to a baseline level of Reelin expression or activity, the level of Reelin expression or activity is changed as compared to the established baseline by an amount that is statistically significant (i.e., with at least a 95% confidence level, or p<0.05). Preferably, detection of at least about a 5% change, and more preferably, at least about a 10% change, and more preferably, at least about a 20% change, and more preferably, at least about a 30% change, and more preferably, at least about a 40% change, and more preferably, at least about a 50% change, in Reelin expression or biological activity in the sample as compared to the baseline level results in a diagnosis of a difference between the test sample and the baseline sample. In one embodiment, a 1.5 fold change in Reelin expression or biological activity in the sample as compared to the baseline level, and more preferably, detection of at least about a 3 fold change, and more preferably at least about a 6 fold change, and even more preferably, at least about a 12 fold change, and even more preferably, at least about a 24 fold change in Reelin expression or biological activity as compared to the baseline level, results in a diagnosis of a significant change in Reelin expression or activity as compared to the baseline sample.

It is to be noted that in some conditions, the levels of individual size forms of Reelin may actually increase in the blood and be indicative of a Reelin deficiency or dysfunction in the brain, for example. In these embodiments, the method is adjusted accordingly. Moreover, for a more sensitive diagnostic or monitoring assay, the individual size forms of Reelin are detected and compared to a baseline control. In this manner, an entire profile of Reelin size forms can be evaluated against a corresponding baseline profile. In this embodiment, certain forms of Reelin may increase in the sample as compared to the baseline, whereas other forms may simultaneously decrease or remain substantially the same. In this embodiment, comparison of the change in Reelin expression or activity and the determination of whether this change indicates a FABP or DHA or other PUFA deficiency in the patient is made by comparison of at least one size form or by comparison of the entire profile to the baseline. Evaluation of the profile of Reelin forms in a patient is described in detail in PCT Publication No. WO 03/063110, which is incorporated herein by reference in its entirety.

Once a positive diagnosis of Reelin deficiency or dysfunction is made using the present method, the diagnosis can be substantiated, if desired, using any suitable alternate method of detection of DHA (or other PUFA) or FABP deficiency or dysfunction.

Treatment of a patient with a diagnosis of Reelin deficiency or dysfunction is provided by administration of PUFA supplementation and in one embodiment, preferably DHA supplementation. The present invention describes the use of Reelin expression and activity to predict a level of DHA in the brain or other tissue of a patient, which is then used to provide an appropriate dosage of DHA and/or other PUFA to compensate for the effects of Reelin deficiency or dysfunction in the patient. The amount of PUFA to be provided to a patient is described above, and can be determined based on the comparison of the patient sample to established control samples, wherein the control samples have been correlated with levels of DHA in the brain or other tissues, and with an amount of PUFA needed to provide a benefit to the patient. Preferred doses of PUFA are discussed above. In one embodiment, a minimum amount of PUFA supplementation is provided to the patient and the patient is reevaluated after an amount of time (e.g., several days, weeks or months) to evaluate the effects of the PUFA supplementation on Reelin expression or activity, or on a symptom or disease or condition associated with Reelin deficiency. If there is no significant change or improvement in the patient, the PUFA supplementation protocol is adjusted upward by the clinician or physician and the patient is reevaluated at a later time point for Reelin expression or activity. In addition to evaluating the amount of PUFA supplementation, the ratio and types of PUFAs to be administered to the patient may be adjusted periodically.

In one embodiment of the invention, a method to identify neural progenitor cells is provided. The method includes detecting Reelin expression and/or biological activity in a population of cells, wherein a defined level of Reelin expression or biological activity is associated with neural progenitor cells. In one embodiment, the method further comprises selecting the neural progenitor cells for which Reelin expression or biological activity was detected.

In another embodiment, the present invention provides a method to monitor neural development, comprising: (a) providing a population of cells comprising neural progenitor cells; (b) detecting Reelin expression or activity in the population of cells; (c) exposing the population of cells to conditions under which the neural progenitor cells will develop into differentiated neural cells; and (d) monitoring the expression or activity of Reelin in the cells after step (c), to evaluate the development of the neural progenitor cells into differentiated neural cells. In this embodiment, the method can include contacting the population of cells of step (a) with a putative developmental regulatory compound prior to or concurrent with step (b), and determining whether the putative regulatory compound affects the development of the neural progenitor cells into differentiated neural cells by detecting Reelin expression or activity in the population of cells.

Detecting Reelin expression or activity in cells can be performed as discussed previously herein. As used herein, the term “putative regulatory compound” refers to compounds having an unknown or previously unappreciated regulatory activity in a particular process. The above-described method for identifying a compound of the present invention includes a step of contacting a test cell with a compound being tested for its ability to regulate the development of neural progenitor cells, using Reelin expression as a marker to track neural cell differentiation and development. For example, test cells can be grown in liquid culture medium or grown on solid medium in which the liquid medium or the solid medium contains the compound to be tested. In addition, the liquid or solid medium contains components necessary for cell growth, such as assimilable carbon, nitrogen and micronutrients.

The above-described methods, in one aspect, involve contacting cells with the compound being tested for a sufficient time to allow for interaction of the putative regulatory compound with an element that affects development in a cell. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which growth is continuing prior to scoring. The conditions under which the cell of the present invention is contacted with a putative regulatory compound, such as by mixing, are any suitable culture or assay conditions and includes an effective medium in which the cell can be cultured or in which the cell can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

According to the present invention, the methods of the present invention are suitable for use in a patient that is a member of the Vertebrate class, Mammalia, including, without limitation, primates, livestock and domestic pets (e.g., a companion animal). Most typically, a patient will be a human patient.

The following examples are provided for the purpose of illustration and are not intended to limit the scope of the present invention.

### EXAMPLES

### Example 1

### Quantitative Determination of Reelin Levels in Infant Patient Samples in Order to Ascertain the Nature of Neurological Dysfunction and Receptiveness to Treatment

The following example demonstrates how a diagnosis of autism and the resulting course of treatment with DHA can be determined by testing patient samples for the concentration of Reelin.

Patient Samples

Patient blood samples are drawn by performing venipuncture or heel sticks on infants ranging from 1 month to 18 months in age. Samples are collected in anticoagulant (EDTA or heparin) containing tubes, and spun down to separate the plasma from the cell pellet. The resulting plasma is frozen at −80° C. until needed.

Control Samples

Blood samples are drawn from suitable, disease-negative control subjects in the same manner as that for the test subjects. The resulting plasma is likewise frozen at −80° C. until needed.

Quantitative Determination of Reelin Levels by Quantitative Western Blotting

Five microliters of each patient's plasma are diluted into SDS-PAGE sample buffer and heated to 95° C. for 10 minutes to fully denature the sample. An appropriate amount of each sample is loaded onto a single lane of a fixed concentration stacking gel on top of a fixed concentration resolving gel. Samples are loaded alongside plasma control samples diluted to multiple known concentrations, as well as appropriate molecular weight markers. The gel is electrophoresed under standard conditions, and the resolved proteins are electroblotted onto nitrocellulose membranes. The resulting blots are blocked for 2 hours at room temperature in PBS containing 1% BSA and 0.1% Tween-20. The buffer is removed and the blots are incubated overnight with blocking buffer containing 5-10 μg/mL of rabbit anti-Reelin IgG antibodies. The following day the blots are washed and then incubated with buffer containing 5-10 μg/mL goat anti-rabbit IgG conjugated to horseradish peroxidase for 1 hour at room temperature. The blots are then washed again and detected with a chemiluminescent substrate exposed to film. Several different molecular weight bands corresponding to different size variants of Reelin are detected in patient and control samples by the anti-Reelin antibodies. Densitometry measurements are taken of the resulting Reelin reactive bands in the patient test samples and known control samples. The quantitative levels of Reelin in the patient samples are then determined by comparison of the densitometry results for these samples to a curve generated by samples containing multiple known concentrations of Reelin.

Analysis

A diagnosis of autism is then made by comparing the levels of the each of the different size forms of Reelin (Reelin moieties) in the patient samples to those in disease-negative control samples. An increase or decrease in the levels of one or more of the forms of Reelin in the patient sample relative to the control samples is indicative of autism in that patient.

Treatment and Monitoring

Based on the levels of Reelin as determined above, a treatment regimen is designed for the patient. Preventive intervention is administered by infant formula supplemented with higher levels of DHA and ARA than in a normal infant formula until the infant reaches 12 months of age (e.g., at a dosage of from about 0.2 g/day to about 1 g/day). Then supplementation is switched to about 1 g of DHA/day provided in a single use tear off capsule until the infant reaches 3 years of age.

Reelin levels are assessed every three months and the dosage is modified accordingly if Reelin levels do not increase to within 85% of mean baseline data.

### Example 2

### Quantitative Determination of Reelin Levels in Patients for the Purpose of Diagnosing Schizophrenia

The following example demonstrates how a diagnosis of schizophrenia and the resulting course of treatment with DHA can be facilitated by quantitatively measuring Reelin levels in peripheral blood samples.

Patient Samples

Blood samples are drawn by performing venipuncture on patients and collecting the samples in anticoagulant (EDTA or Heparin) containing tubes. The samples are spun down to remove the plasma from whole cells and the resulting plasma is frozen at −80° C. until needed.

Control Samples

Blood samples are drawn from suitable, disease-negative control subjects in the same manner as for the test subjects. The resulting plasma is likewise frozen at −80° C. until needed.

Quantitative Determination of Reelin Levels by Fluorescent Microplate Immunoassay

Fifty microliters of each patient's plasma are diluted two-fold in an equal volume of assay buffer consisting of PBS plus 0.5% BSA and 0.05% Tween-20. Control samples containing known concentrations of Reelin are also diluted in assay buffer in a serial fashion in order to construct a known standard curve. The diluted samples and controls are added to individual wells of a black polystyrene microplate that has been coated with a rabbit anti-Reelin N-terminus IgG antibody and then blocked with blocking buffer consisting of PBS plus 1% BSA and 0.1% Tween-20. The anti-Reelin coating antibody used is pan-specific for all three size forms of Reelin that are measured in the assay. The diluted samples are incubated in the microplate wells for 2 hours at 37° C., at which point they are aspirated from the wells and the wells are washed 4 times with wash buffer consisting of PBS plus 0.1% Tween-20. The wells are blotted dry and 100 μL of a mixture of three different rabbit anti-Reelin IgG antibodies, each conjugated to a different fluorescent probe and diluted to 1-10 μg/ml in assay buffer, is added to each well of the plate. Each of the different anti-Reelin detection antibodies is specific for one of the three different size forms of Reelin being measured. The wells are incubated for 1 hour at 37° C., and then washed 4 times with wash buffer. They are then blotted dry and 100 μL of PBS is added to each well. The microplate is then read in a fluorescent microplate reader set up to measure prompt fluorescence using suitable sets of excitation and emission filters for each of the antibody-fluorescent probe conjugates. The emission intensities of each of the fluorescent probes is measured, and by comparing these measurements to those obtained in the known standard curve, the concentration of each size form of Reelin in each patient or control sample can be determined.

Analysis

A diagnosis of schizophrenia is made by comparing the levels of the each of the different size forms of Reelin (Reelin moieties) in the patient samples to those in disease-negative control samples. An increase or decrease in the levels of one or more of the forms of Reelin in the patient sample relative to the control samples is indicative of schizophrenia in that patient.

Treatment and Monitoring

Based on the levels of Reelin as determined above, a treatment regimen is designed for the patient. Therapeutic intervention is accomplished by administering DHA in capsule form at a dosage of 0.2 to 1 g/day. Circulating Reelin levels are then monitored by testing every two months and correlated to clinical symptoms. If Reelin levels do not increase significantly or clinical symptoms do not improve or abate within 6 to 8 months, the dosage of DHA can be increased and further supplemented with other fatty acid compounds, including other n-3 fatty acid precursors.

### Example 3

### Quantitative Determination of Reelin Levels in Patients for the Purpose of Diagnosing a Bipolar Disorder

This example demonstrates how a diagnosis of a bipolar disorder and the resulting course of treatment with DHA can be facilitated by quantitatively measuring Reelin levels in peripheral blood samples.

Patient Samples

Blood samples are drawn by performing venipuncture on patients and collecting the samples in anticoagulant (EDTA or Heparin) containing tubes. The samples are spun down to remove the plasma from whole cells and the resulting plasma is frozen at −80° C. until needed.

Control Samples

Blood samples are drawn from suitable, disease-negative control subjects in the same manner as that for the test subjects. The resulting plasma is likewise frozen at −80° C. until needed.

Quantitative Determination of Reelin Levels Using a Multiwell Fluorescent Protein Microchip Immunoassay

Twenty-five microliters of each patient's plasma are diluted four-fold in 75 mL of assay buffer consisting of PBS plus 0.5% BSA and 0.05% Tween-20. Control samples containing known concentrations of Reelin are also diluted in assay buffer in a serial fashion in order to construct a known standard curve. The diluted samples and controls are added to individual wells attached to a glass slide upon which different rabbit anti-Reelin IgG capture antibodies have been printed in discreet spots. Each well contains multiple individual spots consisting of one of the three capture antibodies specific for the different size forms of Reelin being measured, arrayed out in a two dimensional fashion. In addition to being printed with the individual capture antibodies, each well of the slide is also blocked with PBS plus 1% BSA and 0.1% Tween-20. The diluted samples and controls are incubated in the wells of the slide for 2 hours at 37° C. in a humidified chamber. After this incubation, the wells are aspirated and washed 4 times with wash buffer consisting of PBS plus 0.1% Tween-20. After blotting the wells dry, 100 mL of assay buffer containing 0.5-5 mg/ml of a biotinylated rabbit anti-Reelin IgG antibody, pan-specific for all three size forms of Reelin being measured, is added to each well. The slide then is incubated for 1 hour at 37° C. in a humidified chamber. After the incubation, the wells are aspirated and washed 4 times with wash buffer and blotted dry. At this point, 100 mL of assay buffer containing 10-20 mg/ml of streptavidin conjugated to a fluorescent probe is added to each well. The slide then is incubated for 1 hour at room temperature in a humidified chamber, at which point the wells are aspirated and washed 4 times with wash buffer. The wells are carefully removed from the slide and the entire slide is then rinsed in deionized water and dried under a stream of nitrogen. Once dry, the slide is scanned in a laser-equipped, confocal scanner set up with emission filters suitable for the streptavidin-fluorescent probe conjugate used in the assay. A digital, bitmapped image of the slide is generated and intensities for all spots are determined using microarray image analysis software. By comparing the intensities of each of the individual Reelin spots in the patient sample wells to the corresponding spots in the known standard curve wells, the concentration of each size form of Reelin in each patient or control sample can be determined.

Analysis

A diagnosis of a bipolar disorder is made by comparing the levels of the each of the different size forms of Reelin (Reelin moieties) in the patient samples to those in disease-negative control samples. An increase or decrease in the levels of one or more of the forms of Reelin in the patient sample relative to the control samples is indicative of a bipolar disorder in that patient.

### Treatment and Monitoring

Based on the levels of Reelin as determined above, a treatment regimen can be designed for the patient. Therapeutic intervention can be accomplished by having the patient ingest a food product that is supplemented with DHA in the form of an emulsion at a dosage of 0.2 to 1 g/day. The patient is monitored for psychological or behavioral changes, and blood samples are taken every 3 months to determine circulating Reelin levels. Depending on the patient's continuing psychological and behavioral condition, and their Reelin levels, the therapy can be modified to provide a different dosage of DHA or a different formulation of DHA and other lipids.

### Example 4

This example demonstrates that male, homozygous mutant reeler mice have significantly elevated DHA content in the temporal lobe as compared to wild-type and heterozygous animals or female animals, and that homozygous mutant reeler animals have significantly elevated temporal lobe ARA as compared to wild-type and heterozygous animals.

“Reeler mice” (Reln^{rl}) are mice which are homozygous recessive for the gene that expresses the extracellular signaling glycoprotein, Reelin, and which exhibit a “mutant reeler phenotype” displaying developmental and obvious locomotor deficiencies due to inadequate Reelin levels. Reelin protein may be expressed through various tissues of the body including the brain, liver, kidneys, retina and spinal cord. Since Reelin is a biomarker for DHA levels in the brain and other tissues, a Reelin deficiency can also be corrected through the therapeutic use of DHA.

As set forth in materials from Jackson Laboratories, Bar Harbor, Me., homozygous reeler mice exhibit an ataxic gait, dystonic posture and tremors at about 2 weeks of age. These mutants are incapable of maintaining their hindquarters upright and often fall over during locomotor activity. Viability and fertility are greatly reduced.

Heterozygotes are visually indistinguishable from wildtype controls and therefore genotype assessment must be done to confirm the presence of a reeler gene. The behavioral phenotype is due to the severe hypoplasia of the cerebellum. The following study performed by the present inventors determined whether Reelin can serve as a serum-based biomarker for long chain polyunsaturated fatty acid (LC-PUFA) deficits in the central nervous system.

Specific Aim: To evaluate whether differences in long-chain polyunsaturated fatty acid status are evident in brain tissue from mice with normal or abnormal Reelin expression.

Materials and Methods: Thirty-six animals between the ages of 6 and 12 weeks of age were studied in this experiment. The group contained mice with two copies of the reelin gene mutation (homozygous, n=12); mice with one copy of the reelin gene mutation (heterozygous; n=12), and mice with no mutations in the reelin gene (wild-type; n=12, controls). Within each genotype group, approximately equal numbers of males and females were studied. Homozygous reeler mutant mice were identified by phenotype. Heterozygous reeler mutant mice and normnal wild-type controls were identified by genotypic analysis. Mice were fed normal rodent chow during the study.

Mouse Brain Tissue Fatty Acid Analysis: Mouse brain tissue was analyzed for fatty acid content directly. Total lipids in the sample were saponified and converted to fatty acid methyl esters before analysis. Briefly, mouse temporal lobes were kept frozen at −80° C. until analysis. Samples were lyophilized prior to analysis. The lyophilized sample was weighed directly into a screw cap test tube and pulverized using a glass rod. 1.0 mL of toluene containing internal standard (methyl nonadecanoate was added to the sample along with 1.0 mL of 0.5 N NaOH. The tube was purged with nitrogen, capped, and heated at approximately 100° C. for approximately 5 minutes in a heat block. The tube was removed and allowed to cool. Two mL of 14% BF₃ in methanol was added to the tube, the tube was purged with nitrogen, and capped. The tube was heated to approximately 100° C. for approximately 30 minutes in a heat block. After 30 minutes, the tube was removed and allowed to cool. One milliliter of aqueous saturated sodium chloride solution was added to the tube and the tube was vortexed. The layers were allowed to separate and a portion of the organic (top) layer was removed for analysis. Fatty acid methyl esters were analyzed by gas-liquid chromatography with flame ionization detection (GLC-FID) on an Agilent Technologies gas chromatograph (model 5890) equipped with a flame ionization detector. The fatty acid methyl esters were separated on a 30 meter FAMEWAX capillary column (Restek, Bellefonte, Pa.; 0.25 mm diameter, 0.25 μm coating thickness) using helium at a flow rate of 2.0 mL/min with a split ratio of 15:1. The chromatographic run parameters included an oven starting temperature of 130° C. that was increased at 5° C./min to 225° C., where it was held for 20 minutes before increasing to 250° C. at 15° C./min, with a final hold of 5 minutes. The injector and detector temperatures were constant at 220° C. and 230° C. respectively. Peaks were identified by comparison of retention times with fatty acid methyl ester standard mixtures from NuCheck Prep (Elysian, Minn., U.S.A.). Individual fatty acids were expressed as a percent of the total fatty acids present (weight percent).

Data were analyzed by 2-way General Linear Model ANOVA with p<0.05. When interactions were present, significant differences between means were assessed by t-test.

### Results:

### Docosahexaenoic Acid Content of the Temporal Lobe

Main Effects Data for temporal lobe DHA content is shown in Table 1. There were no significant main effect differences in DHA fatty acid composition of the temporal lobes of mice with different capabilities for reelin expression (P=0.406). There were no differences in DHA fatty acid composition of the temporal lobes of male or female mice (P=0.267). However, significant group and gender interactions were evident (P=0.019), allowing specific statistical comparison of each genotype-sex subgroup. This comparison showed that highest levels of temporal lobe DHA were evident in homozygous male reelers, and lowest levels of DHA were present in homozygous female reelers (P=0.006).

Homozygous male reeler mice had significantly greater temporal lobe DHA content compared to heterozygous males but not compared to wild-type males. Temporal lobe DHA content of homozygous female animals did not significantly differ by genotype.

TABLE 1
Temporal lobe DHA content (wt % of total fatty acids)

| | Wild Type | Homozygous | Heterozygous |
|---|---|---|---|
| All | 19.51 ± 0.24 | 19.62 ± 0.21 | 19.29 ± 0.15 |
| Female | 19.62 ± 0.39^{ab} | 19.10 ± 0.25^{a} | 19.34 ± 0.29^{ab} |
| Male | 19.40 ± 0.31^{ab} | 20.18 ± 0.12^{b} | 19.22 ± 0.11^{a} |

Note:
mean ± sem indicates that different superscripts are significantly different at p < 0.05; in the case of male animals, the DHA content in the temporal lobe of the wild type animals was lower than in the homozygous animals but did not reach the level of significance specified for this study. (p = 0.055). Homozygous females are different than homozygous males with p < 0.05.

### Conclusion:

Male, homozygous mutant reeler mice have significantly elevated DHA content in the temporal lobe.

### Results:

### Arachidonic Acid Content of the Temporal Lobe

Main Effects Significant differences in temporal lobe DHA content were evident between mice of different genotypes (P=0.004). Homozygous reeler animals had significantly more temporal lobe ARA compared to wild-type animals (P<0.001), but similar temporal lobe ARA compared to heterozygous animals. Temporal lobe ARA content of heterozygous mice was greater than in wild-type animals, but did not reach the criteria for statistical significance (P=0.061). There were no significant differences in ARA content of temporal lobe between male and female animals.

Interaction effects: There were no significant interactions between genotype and gender.

TABLE 2
Temporal Lobe Arachidonic Acid content (wt % of total fatty acids)

| | Wild Type | Homozygous | Heterozygous |
|---|---|---|---|
| All | 9.43 ± 0.16^{a} | 10.26 ± 0.21^{b} | 9.87 ± 0.16^{ab} |
| Female | 9.37 ± 0.15 | 10.05 ± 0.27 | 9.77 ± 0.31 |
| Male | 9.50 ± 0.29 | 10.48 ± 0.16 | 9.98 ± 0.06 |

Data are mean ± sem. Homozygous mutant reeler animals have significantly elevated temporal lobe ARA compared to the wild-type or heterozygous groups. There were no significant differences in the ARA content of temporal lobe between male and female animals.

Conclusion: Homozygous mutant reeler animals have significantly elevated temporal lobe ARA.

### Example 5

The following example demonstrates the relationship between Reelin and red blood cell HUFA status. Specifically, the inventors determined whether animals with different levels of reelin expression will manifest different DHA and ARA content in red blood cells.

Materials and Methods: (Same as above in Example 4).

Results:

TABLE 3
Gender/Red Blood Cell Fatty Acid Content (wt % of total fatty
acids)

| Genotype | Gender | (n) | DHA | (n) | ARA |
|---|---|---|---|---|---|
| Control | Female | 6 | 4.92 ± 0.39^{a} | 6 | 6.40 ± 0.49^{ab} |
| Control | Male | 6 | 6.15 ± 0.20^{a} | 6 | 6.98 ± 0.36^{a} |
| Control | All | 12 | 5.54 ± 0.28^{a} | 12 | 6.54 ± 0.32^{a} |
| Homo | Female | 6 | 5.13 ± 0.64^{a} | 6 | 6.72 ± 0.76^{b} |
| Homo | Male | 6 | 6.13 ± 0.32^{ac} | 6 | 7.84 ± 0.28^{a} |
| Homo | All | 12 | 5.63 ± 0.37^{a} | 12 | 7.29 ± 0.42^{a} |
| Hetero | Female | 6 | 3.66 ± 0.35^{bc} | 6 | 4.61 ± 0.40^{b} |
| Hetero | Male | 5 | 5.86 ± 0.49^{a} | 5 | 6.52 ± 0.67^{a} |
| Hetero | All | 11 | 4.66 ± 0.44^{a} | 12 | 5.56 ± 0.47^{a} |

Means in each column with unlike superscripts differ significantly (P < 0.05)

### Summary:

### RBC DHA

Main Effects: No statistically significant differences in RBC DHA content were observed between animals with different genotypes. Male animals had significantly higher RBC DHA content than female animals (6.06±0.76 vs 4.57±1.29%).

Interaction Effects: No interaction was detected for RBC DHA content between genotype and gender variables.

Conclusion: DHA content of RBC does not differ in mice differing in reelin status. DHA content of RBC from males is higher than DHA content of RBC from females.

### RBC ARA

Main Effects: Statistically significant differences in RBC ARA content were observed between animals with different genotypes (P<0.01) and gender (P<0.005). Mice with 2 copies of the mutant reelin gene had significantly lower levels of RBC ARA compared to mice with 1 copy of the mutant reelin gene. RBC ARA content of wild-type controls did not differ significantly from mice with one or two copies of the mutant reelin gene. Male animals had significantly higher RBC ARA compared to female animals.

Interaction Effects: No interaction was detected for RBC ARA content between genotype and gender variables.

Conclusion: RBC ARA content of mice is modified by reelin status. Mice with low reelin status tend to have low RBC levels of ARA. Male animals tend to have significantly higher RBC ARA than females.

### Example 6

The following example demonstrates that providing DHA to mice with abnormal reeler gene expression can reduce the number of male offspring with reeler phenotypic symptoms. In the following experiment, the inventors tested whether LC-PUFA dietary enrichment (DHA) for mice lacking one or more normal reelin genes will correct Reelin histopathology/symptoms and will normalize the fatty acid profiles observed in Reelin-deficient mice. Specifically, the inventors evaluated whether dietary enrichment of long-chain polyunsaturated fatty acids (DHA) will correct or modulate Reelin histopathology/symptoms in Reelin-deficient mice.

Materials and Methods: The Reelin feeding study was sponsored by Martek Biosciences Corporation and initiated at Jackson Labs, Bar Harbor, Me. (Stock used: 300235 B6C3Fe a/a-Reln<rl>/J). Heterozygous females were mated with heterozygous males and received one of two experimental diets: a DHA DEFICIENT DIET (0% DHA by weight; 0.14% alpha linolenic acid by weight), or a DHA ADEQUATE DIET (0.462% DHA by weight, with 0.115% alpha linolenic acid by weight). The females continued to receive the specific diet throughout pregnancy and lactation. Homozygous, heterozygous, and wild-type pups born to pregnant females were placed on the same specific maternal diet at weaning. The number of reeler mice was recorded within each diet group. Pups that did not exhibit the reeler phenotype were genotyped for confirmation of their reeler gene status. Pups were sacrificed at between 8 and 14 weeks of age and tissues were collected for fatty acid analysis.

Results:

### DHA Adequate Diet:

Out of 94 pups born, 14 mice (10F, 4M) were observed to have a reeler phenotype (14.8%). Four males out of 40 (10%) exhibited reeler phenotype. Ten females out of 54 (18.5%) exhibited reeler phenotype.

### DHA Deficient Diet:

Out of 89 pups born, 19 mice (8F, 11M) were observed to have reeler phenotype (or 21.3%. Eleven males out of 40 (27.5%) exhibited the reeler phenotype, while eight females out of 48 (16.6%) exhibited reeler phenotype.

Chi-square analysis revealed that significantly fewer reeler mice were born in the DHA Adequate Diet group compared to the DHA Deficient Diet group. Moreover, a chi-square analysis to detect incidence of male reeler mice showed that the provision of DHA to the pregnant and lactating dam and to the pups after weaning reduced the incidence of male reeler animals by almost 3-fold (P=0.04). A total of 11 males out of 40 total males, or 27.5% of males in the DHA Deficient Diet exhibited the reeler phenotype, whereas only 4 out of 40 total males, or 10% of males in the DHA Adequate Diet exhibited the reeler phenotype.

TABLE 4
DHA Adequate and DHA Deficient Diets vs % Reeler Phenotypes

| | Avg | | Male Reeler | | |
|---|---|---|---|---|---|
| | Days | Pups | Mice | Female Reeler | Total % |
| | Feeding | Born | Phenotype % | Mice Phenotype % | Reeler Mice |
| DHA Adequate | 28 | 94 | 4/40 (10%) | 10/54 (18.5%) | 14/94 (14.8%) |
| Diet | | | | | |
| DHA Deficient | 27 | 89 | 11/40 (27.5%) | 8/48 (16.6%) | 19/89 (21.3%) |
| Diet | | | | | |

The incidence of male reeler mice born to DHA supplemented dams was significantly lower than the incidence of male reeler mice born to DHA deficient dams. The total incidence of reeler mice (males plus females) did not sign differ between dietary groups.

Conclusion: Supplementation of DHA to reelin-deficient mice during pregnancy can substantially reduce the number of male offspring with reeler phenotypes.

### Example 7

### Modulation of Red Blood Cell Fatty Acid Content by Dietary DHA

The following example shows the changes in red blood cell DHA and ARA in mice differing in Reelin status and dietary DHA exposure. Specifically, the inventors determined whether dietary content can correct the differences in fatty acid composition of RBC in mice with different Reelin status. Since the inventors show above that male mice with mutant reelin gene expression tend to have abnormally high RBC ARA content, it was determined whether DHA supplementation could modulate ARA expression in RBCs of male mice with mutant reelin genes.

Materials and Methods: The Reelin feeding study was sponsored by Martek Biosciences Corp. and initiated at Jackson Labs, Bar Harbor, Me. (Stock used: 300235 B6C3Fe a/a-Reln<rl>/J). Heterozygous females were mated with heterozygous males and received one of two experimental diets: a DHA DEFICIENT DIET (0% DHA by weight; 0.14% alpha linolenic acid by weight), or a DHA ADEQUATE DIET (0.462% DHA by weight, with 0.115% alpha linolenic acid by weight). The females continued to receive the specific diet throughout pregnancy and lactation. Homozygous, heterozygous, and wild-type pups born to pregnant females were placed on the same specific maternal diet at weaning. Thirty-six animals between the ages of 6 and 12 weeks of age were studied in this experiment. The group contained mice with two copies of the reelin gene mutation (homozygous, n=12); mice with one copy of the reelin gene mutation (heterozygous; n=12), and mice with no mutations in the reelin gene (wild-type; n=12, controls). Within each genotype group, approximately equal numbers of males and females were studied. The number of reeler mice was recorded within each diet group. Pups that did not exhibit the reeler phenotype were genotyped for confirmation of their reeler gene status. Pups were sacrificed at between 8 and 14 weeks of age and tissues were collected for fatty acid analysis.

Mouse Red Blood Cell Analysis of Fatty Acids: Mouse red blood cells (RBCs) were extracted and analyzed for fatty acid content. Total lipids in the sample were saponified and converted to fatty acid methyl esters before analysis. Briefly, RBCs were kept frozen at −80° C. until analysis. Fifty microliters of chloroform containing internal standard (methyl tricosanoate) was added to a screw cap test tube. The chloroform was evaporated under a stream of nitrogen. Approximately 300 microliters of sample was added to the internal standard along with 1.5 mL of 1:2 chloroform:methanol. The tube was capped and vortexed for approximately 30 seconds. The tube was placed in ice in a sonicating bath for approximately 20 minutes. After 20 minutes the tube was removed and one milliliter of chloroform and one milliliter of water was added to the tube. The tube was vortexed for approximately 30 seconds and centrifuged at approximately 2000 rpm for approximately 10 minutes. The bottom layer was removed to another screw cap test tube, and the solvent evaporated under nitrogen. One milliliter of toluene was added to the sample along with 1.0 mL of 0.5 N NaOH. The tube was purged with nitrogen, capped, and heated at approximately 100° C. for approximately 5 minutes in a heat block. The tube was removed and allowed to cool. Two mL of 14% BF₃ in methanol was added to the tube, the tube was purged with nitrogen, and capped. The tube was heated to approximately 100° C. for approximately 30 minutes in a heat block. After 30 minutes, the tube was removed and allowed to cool. One milliliter of aqueous saturated sodium chloride solution was added to the tube and the tube was vortexed. The layers were allowed to separate and a portion of the organic (top) layer was removed for analysis. Fatty acid methyl esters were analyzed by gas-liquid chromatography with flame ionization detection (GLC-FID) on an Agilent Technologies gas chromatograph (model 5890) equipped with a flame ionization detector. The fatty acid methyl esters were separated on an 30 meter FAMEWAX capillary column (Restek, Bellefonte, Pa.; 0.25 mm diameter, 0.25 μm coating thickness) using helium at a flow rate of 2.0 mL/min with a split ratio of 15:1. The chromatographic run parameters included an oven starting temperature of 130° C. that was increased at 5° C./min to 225° C., where it was held for 20 minutes before increasing to 250° C. at 15° C./min, with a final hold of 5 minutes. The injector and detector temperatures were constant at 220° C. and 230° C. respectively. Peaks were identified by comparison of retention times with fatty acid methyl ester standard mixtures from NuCheck Prep (Elysian, Minn., U.S.A.). Individual fatty acids were expressed as a percent of the total fatty acids present (weight percent).

### Results:

DHA

Animals fed a diet deficient in preformed DHA had significantly lower levels of RBC DHA than animals fed a diet containing preformed DHA at 0.5% by weight. Homozygotes and male heterozygotes were more susceptible to the effects of dietary DHA deficiency, since these animals had significantly lower RBC DHA compared to wildtype control males and females and heterozygous female animals fed a fed a DHA deficient diet. Addition of DHA to the diet significantly increased RBC DHA levels in all groups. However, DHA supplemented diets did not fully restore RBC DHA levels in female animals with mutant reelin genes to levels observed in wild-type control animals. Specifically, homozygous females fed a DHA supplemented diet had significantly lower RBC DHA compared to all other genotype/gender subgroups fed a DHA adequate diet. Heterozygous females fed a DHA supplemented diet had significantly lower RBC DHA than wild-type control females. The DHA supplemented diet restored RBC DHA levels in male heterozygous and homozygous animals to similar levels observed in wild-type males.

TABLE 5
Red Blood Cell/DHA Content (wt % in total fatty acids)

| | | MEAN ± SD | | MEAN ± SD | |
|---|---|---|---|---|---|
| | | Yellow | | Green | |
| | | DHA | | DHA | |
| | | Deficient Diet | (n) | Adequate Diet | (n) |
| Control | Female | 4.06 ± 0.34^{a} | 3 | 11.20 ± 0.21^{c} | 4 |
| Control | Male | 3.60 ± 0.06^{a} | 3 | 10.92 ± 0.45^{cd} | 3 |
| Heterozygous | Female | 3.71 ± 0.06^{a} | 3 | 10.12 ± 0.25^{d} | 3 |
| Heterozygous | Male | 3.29 ± 0.10^{b} | 3 | 10.29 ± 0.52^{cd} | 3 |
| Homozygous | Female | 3.20 ± 0.13^{b} | 3 | 7.99 ± 0.47^{e} | 5 |
| Homozygous | Male | 2.94 ± 0.30^{b} | 3 | 10.23 ± 1.17^{cd} | 3 |

^{a} vs ^{b}P < 0.05
^{a} vs ^{c,d,} or ^{e}P < 0.0001
^{b} vs ^{c,d} or ^{e}P < 0.001
^{c} vs ^{d}P < 0.05
^{c} or ^{d} vs ^{e}P < 0.0001
^{d} vs ^{e}P < 0.0001

Conclusion: Red Blood Cell DHA content in female animals with 1 or 2 copies of the reelin gene cannot be fully normalized by feeding a diet containing 0.5% DHA by weight. RBC DHA content in male animals with 1 or 2 copies of the reelin gene is modulated in a similar manner as wildtype control animals when fed a diet containing 0.5% DHA by weight.

### ARA

Red Blood Cell ARA levels of animals fed a diet with no preformed DHA were significantly greater than RBC ARA levels of animals fed a diet containing 0.5% DHA by weight. Within animals fed the diet deficient in preformed DHA, RBC ARA levels of wildtype control and heterozygous reeler mice were significantly greater than detected in homozygous reeler mice. Feeding a diet containing 0.5% DHA suppressed RBC ARA levels by approximately 2-fold and eliminated differences in RBC ARA levels between genotype subgroups. No significant differences in RBC ARA levels were detected between male and female animals within or between genotype subgroups when animals were fed 0.5% DHA by weight.

TABLE 6
Red Blood Cell/ARA Content (wt % in total fatty acids)

| | RBC ARA (wt %) | | | |
|---|---|---|---|---|
| | Control | Heterozygous | Homozygous | |
| DHA | 15.72 ± 0.48^{b} | 14.89 ± 1.47^{b} | 12.51 ± 1.77^{c} | a vs b |
| Deficient | | | | P < |
| Diet | | | | 0.0001 |
| (n) | 6 | 6 | 5 | a vs c |
| | | | | P < |
| | | | | 0.0001 |
| DHA | 6.84 ± 0.61^{a} | 6.45 ± 0.77^{a} | 6.14 ± 0.95^{a} | b vs c |
| Adequate | | | | P < 0.001 |
| Diet | | | | |
| (n) | 7 | 6 | 6 | |

Mean ± sem

Conclusions: Animals that receive no preformed dietary DHA tend to have high levels of RBC ARA. Low reelin expression is associated with lower RBC DHA content. Dietary DHA suppresses ARA incorporation into the RBC membrane and equalized RBC ARA content in wildtype controls and animals with lower reelin expression (i.e. heterozygotes and homozygotes).

Each reference and publication cited herein is incorporated by reference in its entirety. Each of U.S. Provisional Application No. 60/537,600, filed Jan. 19, 2004, and U.S. Provisional Application No. 60/605,219, filed Aug. 27, 2004, is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method to treat a Reelin deficiency or dysfunction, comprising administering to a patient diagnosed with or suspected of having a Reelin deficiency or dysfunction an amount of a polyunsaturated fatty acid (PUFA) selected from the group consisting of an omega-3 PUFA and an omega-6 PUFA, or a precursor or source thereof, to compensate for the effects of Reelin deficiency or dysfunction in the patient.

2. The method of claim 1, wherein the Reelin deficiency or dysfunction is associated with a decrease in the expression or function of a fatty acid binding protein in the patient.

3. The method of claim 2, wherein the fatty acid binding protein is a brain lipid binding protein (BLBP).

4. The method of claim 1, wherein administration of the PUFA to the patient compensates for reduced fatty acid binding protein or function thereof in the patient.

5. The method of claim 1, wherein administration of the PUFA to the patient compensates for reduced brain lipid binding protein or function thereof in the patient.

6. The method of claim 1, wherein administration of the PUFA to the patient improves the activity of fatty acid binding proteins in the patient.

7. The method of claim 1, wherein administration of the PUFA to the patient improves at least one parameter of the mechanism of action of brain lipid binding proteins in the patient.

8. The method of claim 1, wherein administration of the PUFA to the patient results in increased incorporation of functional DHA into the phospholipid membranes of glial cells and neurons in the patient.

9. The method of claim 1, wherein administration of the PUFA to the patient increases the level of Reelin or improves the activity of Reelin in the patient.

10. The method of claim 1, wherein the patient suffers from a disease or condition associated with the Reelin deficiency or dysfunction, and wherein administration of the PUFA to the patient improves at least one symptom of the disease or condition.

11. The method of claim 1, wherein the patient is at risk of developing a disease or condition associated with the Reelin deficiency or dysfunction, and wherein administration of the PUFA to the patient prevents or delays the onset of the disease or condition.

12. The method of claim 1, wherein, prior to the step of administering, the method comprises measuring an amount or a biological activity of Reelin in a biological sample from the patient.

13. The method of claim 12, further comprising comparing the amount of Reelin in the patient sample to a baseline amount of Reelin in a sample of the same type, wherein a change in the amount of Reelin in the patient sample as compared to the baseline amount indicates that the patient has a Reelin deficiency.

14. The method of claim 12, wherein the step of measuring is performed by a method selected from the group consisting of: mRNA transcription analysis, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and protein microchip or microarray.

15. The method of claim 12, further comprising determining the relative expression or activity of different Reelin size forms in the patient to establish a Reelin size form profile in the patient sample, and comparing the patient Reelin size form profile to a baseline profile of Reelin size forms in a sample of the same type, wherein a change in expression of one or more size forms of Reelin as compared to relative expression or activity of the size forms in the baseline profile indicates that the patient has a Reelin deficiency or dysfunction.

16. The method of claim 15, wherein the step of measuring is performed using a technique selected from the group consisting of: mRNA transcription analysis, Western blot, immunoblot, and capillary electrophoresis.

17. The method of claim 12, further comprising comparing the activity of Reelin in the patient sample to a baseline activity of Reelin in a sample of the same type, wherein a change in the level of activity of Reelin in the patient sample as compared to the baseline level indicates that the patient has a Reelin dysfunction.

18. The method of claim 17, wherein the step of measuring the activity is by a technique selected from the group consisting of: a receptor-ligand assay and a phosphorylation assay.

19. The method of claim 12, further comprising measuring the levels of thyroid stimulating hormone (TSH) in the patient sample and comparing the amount of TSH in the patient sample to a baseline amount of TSH in a sample of the same type, wherein a change in the amount of TSH in the patient sample as compared to the baseline amount indicates that the patient has a TSH deficiency.

20. The method of claim 19, further comprising administering a thyroid medication in conjunction with the PUFA, to the patient.

21. The method of claim 12, wherein the biological sample is selected from the group consisting of a cell sample, a tissue sample, and a bodily fluid sample.

22. The method of claim 21, wherein the biological sample is a blood sample.

23. The method of claim 1, further comprising monitoring the efficacy of the administration of the PUFA on Reelin levels or biological activity in the patient at least one time subsequent to the step of administering.

24. The method of claim 1, further comprising monitoring the efficacy of the administration of the PUFA on changes in the expression or biological activity of one or more size forms of Reelin in the patient at least one time subsequent to the step of administering.

25. The method of claim 23, further comprising adjusting the administration of the PUFA to the patient in subsequent treatments based on the results of the monitoring of efficacy of the treatment.

26. The method of claim 1, wherein the patient has, is suspected of having, or is at risk of developing, a neurological disorder or neuropsychiatric disorder.

27. The method of claim 1, wherein the patient suffers from seizures.

28. The method of claim 1, wherein the patient has, is suspected of having, or is at risk of developing, an autoimmune disorder associated with a neurological dysfunction.

29. The method of claim 1, wherein the patient has an anti-phospholipid disorder.

30. The method of claim 1, wherein the patient has, is suspected of having, or is at risk of developing, a disorder selected from the group consisting of: schizophrenia, bipolar disorder, dyslexia, dyspraxia, attention deficit hyperactivity disorder (ADHD), epilepsy, autism, Parkinson's Disease, senile dementia, Alzheimer's Disease, peroxisomal proliferator activation disorder (PPAR), multiple sclerosis, diabetes-induced neuropathy, macular degeneration, retinopathy of prematurity, Huntington's Disease, amyotrophic lateral sclerosis (ALS), retinitis pigmentosa, cerebral palsy, muscular dystrophy, cancer, cystic fibrosis, neural tube defects, depression, Zellweger syndrome, Lissencepahly, Down's Syndrome, Muscle-Eye-Brain Disease, Walker-Warburg Syndrome, Charoct-Marie-Tooth Disease, inclusion body rnyositis (IBM) and Aniridia.

31. The method of claim 1, wherein the patient has a thyroid disorder.

32. The method of claim 1, wherein the PUFA is administered to the patient in combination with one or more additional therapeutic compounds for treating a condition associated with a Reelin deficiency or dysfunction.

33. A method of modulating Reelin expression in tissues or fluids, comprising administering to a patient an amount of a polyunsaturated fatty acid (PUFA) selected from the group consisting of an omega-3 PUFA and an omega-6 PUFA, or a precursor or source thereof, effective to modulate Reelin expression in a tissue or fluid of the patient.

34. The method of claim 33, wherein the amount of the PUFA is sufficient to increase Reelin expression in a tissue or fluid of the patient.

35. A method to prevent, reduce or delay the onset of retinal developmental defects or disorders, comprising administering to the patient a polyunsaturated fatty acid (PUFA) selected from the group consisting of an omega-3 PUFA and an omega-6 PUFA, or a precursor or source thereof, effective to prevent, reduce or delay the onset of retinal developmental defects or disorders and to compensate for the effects of Reelin deficiency or dysfunction in the patient.

36. A method to prevent, reduce or delay the onset of developmental defects or disorders associated with Reelin deficiency or dysfunction, comprising:
a) measuring the expression or biological activity of Reelin in a biological sample from a patient;
b) administering to the patient a polyunsaturated fatty acid (PUFA) selected from the group consisting of an omega-3 PUFA and an omega-6 PUFA, or a precursor or source thereof, wherein the amount of the PUFA administered is determined based on the measurement of expression or biological activity of the Reelin in the sample.

37. The method of claim 36, wherein the step of measuring the expression or activity of Reelin further comprises determining the relative expression or activity of individual size forms of Reelin in the sample.

38. The method of claim 36, wherein the amount of PUFA administered to the patient is determined by comparing the level of expression or biological activity of Reelin in the patient sample to a baseline level of Reelin expression or activity that corresponds to a recommended dosage of the PUFA, and adjusting the dosage of the PUFA for the patient accordingly.

39. The method of claim 38, wherein the amount of PUFA administered to the patient is increased relative to the recommended dosage of PUFA when the expression or biological activity of Reelin in the patient is decreased relative to the baseline level.

40. The method of claim 36, wherein the amount of PUFA administered to the patient is determined by comparing the expression or activity of different Reelin size forms in the patient sample to a baseline profile of Reelin size forms that corresponds to a recommended dosage of PUFA, and adjusting the dosage of the PUFA for the patient accordingly.

41. The method of claim 40, wherein the amount of PUFA administered to the patient is increased relative to the recommended dosage of PUFA when the relative expression or activity of one or more Reelin size forms in the patient sample differs from the relative expression or activity of the Reelin size form in the baseline profile.

42. The method of claim 36, wherein the step of measuring the expression or biological activity of Reelin in a biological sample from the patient is repeated one or more times subsequent to the administration of the PUFA to the patient.

43. The method of claim 42, wherein the amount of PUFA administered to the patient is adjusted according to the repeated measurement of the expression or biological activity of Reelin in the patient.

44. The method of claim 36, wherein the step of measuring the expression or biological activity of Reelin in a biological sample from the patient is repeated intermittently throughout a portion of the life of the patient or throughout the entire life of the patient, and wherein the amount of PUFA administered to the patient is adjusted to correspond to each new measurement of the expression or biological activity of Reelin in the patient.

45. The method of claim 36, wherein the expression or biological activity of Reelin in the patient is substantially normal, and wherein the PUFA is administered as a supplement to prevent or reduce the risk of development of Reelin deficiency or dysfunction.

46. The method of claim 36, wherein the patient is a pregnant female.

47. The method of claim 36, wherein the patient is a lactating female.

48. The method of claim 36, wherein the patient is a human adult.

49. The method of claim 36, wherein the patient is a human child or adolescent.

50. The method of claim 36, wherein the patient is a human embryo or fetus and wherein the PUFA is administered to the embryo or fetus by administering the PUFA to the mother of the embryo or fetus.

51. The method of claim 36, wherein the patient has or is at risk of developing a neurological disorder or neuropsychiatric disorder associated with Reelin deficiency or dysfunction or a fatty acid binding protein deficiency.

52. The method of claim 36, wherein the patient has or is at risk of developing an autoimmune disease associated with Reelin deficiency or dysfunction or a fatty acid binding protein deficiency.

53. The method of claim 36, wherein the patient has or is at risk of developing a developmental defect associated with Reelin deficiency or dysfunction or a fatty acid binding protein deficiency.

54. A method to monitor the levels of DHA in the brain of a patient, comprising measuring the levels of Reelin expression or biological activity in a biological sample from the patient and estimating the levels of DHA in the brain of the patient based on the measurement of Reelin.

55. The method of claim 54, further comprising administering an amount of DHA to the patient corresponding to the measured levels of Reelin expression or biological activity.

56. The method of claim 55, wherein the amount of DHA administered is sufficient to compensate for reduced expression or activity of brain lipid binding proteins in the patient or to improve the activity of brain lipid binding proteins in the patient.

57. The method of claim 54, further comprising comparing the level of Reelin expression or biological activity in the biological sample from the patient to a baseline level of Reelin expression or biological activity, wherein the baseline level of Reelin expression or biological activity is correlated with a baseline level of DHA in the brain of a subject, wherein the baseline level is established by a method selected from the group consisting of:
a) establishing a baseline level of Reelin expression or activity from a previous measurement of Reelin expression or activity in a previous sample from the patient, wherein the previous sample was of a same cell type, tissue type or bodily fluid type; and,
b) establishing a baseline level of Reelin expression or activity from control samples of a same cell type, tissue type or bodily fluid type as the sample from the patient, the control samples having been obtained from a population of matched individuals.

58. The method of claim 57, wherein an estimated low level of DHA in the brain of the patient as compared to the baseline level of DHA indicates that the patient should be administered an amount of DHA to compensate for the level of DHA in the brain of the patient.

59. A method to predict the efficacy of incorporation of HUFA into the phospholipid membranes in a patient, comprising:
a) measuring Reelin expression or biological activity in a biological sample from a patient;
b) comparing the Reelin expression or biological activity in the biological sample to a baseline level of Reelin; and
c) predicting the patient efficacy of the incorporation of HUFA into phospholipids membranes, wherein a difference in the level of Reelin expression or biological activity in the biological sample as compared to the baseline level of Reelin expression or biological activity indicates a modification in the predicted ability of the patient to efficaciously incorporate HUFA into phospholipids membranes.

60. The method of claim 59, further comprising prescribing an amount of HUFA to the patient, wherein the amount is determined based on the predicted ability of the patient to efficaciously incorporate HUFA into phospholipids membranes.

61. A method to diagnose a DHA deficiency in a patient, comprising:
a) measuring Reelin expression or biological activity in a biological sample from a patient;
b) comparing the Reelin expression or biological activity in the biological sample to a baseline level of Reelin; and,
c) making a diagnosis of the patient, wherein detection of a difference in the level of Reelin expression or biological activity in the biological sample as compared to the baseline level of Reelin expression or biological activity, indicates a positive diagnosis of DHA deficiency in the patient.

62. The method of claim 61, wherein detection of a lower level of Reelin expression or biological activity in the biological sample as compared to the baseline level of Reelin expression or biological activity, indicates a positive diagnosis of DHA deficiency in the patient.

63. The method of claim 61, wherein the biological sample is selected from the group consisting of a cell sample, a tissue sample, and a bodily fluid sample.

64. The method of claim 63, wherein the biological sample is a blood sample.

65. The method of claim 61, wherein the step (a) of measuring comprises measuring Reelin mRNA transcription.

66. The method of claim 65, wherein the step (a) of measuring is by a method selected from the group consisting of reverse transcriptase-PCR (RT-PCR), in situ hybridization, Northern blot, sequence analysis, microarray analysis, and detection of a reporter gene.

67. The method of claim 61, wherein the step (a) of measuring comprises measuring Reelin protein expression.

68. The method of claim 67, wherein the step (a) of measuring is by a method selected from the group consisting of immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting, flow cytometry, and protein microchip or microarray.

69. The method of claim 61, wherein the step (a) of measuring comprises measuring Reelin biological activity.

70. The method of claim 69, wherein the step (a) of measuring is by a method selected from the group consisting of a receptor-ligand assay and a phosphorylation assay.

71. The method of claim 61, wherein the baseline level is established by a method selected from the group consisting of:
a) establishing a baseline level of Reelin expression or activity in an autologous control sample from the patient, wherein the autologous sample is of a same cell type, tissue type or bodily fluid type as the sample of step (a);
b) establishing a baseline level of Reelin expression or activity that is an average from at least two previous measurements of Reelin expression or activity in a previous sample from the patient, wherein each of the previous samples were of a same cell type, tissue type or bodily fluid type as the sample of step (a), and wherein the previous measurements resulted in a negative diagnosis; and,
c) establishing a baseline level of Reelin expression or activity from control samples of a same cell type, tissue type or bodily fluid type as the sample of step (a), the control samples having been obtained from a population of matched individuals.

72. A method to supplement PUFAs in a female during pregnancy and lactation, comprising:
a) measuring the expression or biological activity of Reelin in a biological sample from one or both parents of a fetus or child;
b) administering a polyunsaturated fatty acid (PUFA) selected from the group consisting of an omega-3 PUFA and an omega-6 PUFA, or a precursor or source thereof to the mother of the fetus or child, wherein the amount of PUFA administered is determined based on the measurement of expression or biological activity of the Reelin in the sample from the parent, wherein the PUFA supplements the PUFA in the female and her fetus or child.

73. The method of claim 72, wherein the PUFA is administered in an amount sufficient to compensate for reduced expression or activity of brain lipid binding proteins in the fetus or child or to improve the activity of brain lipid binding proteins in the fetus or child.

74. The method of claim 72, wherein the PUFA is administered in an amount sufficient to decrease the risk of giving birth to an infant with a Reelin deficiency or dysfunction.

75. The method of claim 72, wherein the PUFA is administered in an amount sufficient to decrease the risk of giving birth to a male infant with a Reelin deficiency or dysfunction.

76. The method of claim 72, wherein the PUFA is administered in an amount sufficient to prevent, delay the onset of, or reduce the symptoms of autism in the mother, child or fetus.

77. The method of claim 72, wherein the PUFA is administered in an amount sufficient to prevent, delay the onset of, or reduce the symptoms of neuronal migration disorders in the mother, child or fetus.

78. The method of claim 72, wherein the PUFA is administered in an amount sufficient to prevent, delay the onset of, or reduce the symptoms associated with Reelin deficiency or dysfunction in the mother, child or fetus.

79. A method to supplement PUFAs in a female during pregnancy and lactation to decrease the risk of birth of infants having or at risk of developing a Reelin deficiency or dysfunction, comprising:
a) identifying the gender of the fetus carried by a pregnant female;
b) administering a polyunsaturated fatty acid (PUFA) selected from the group consisting of an omega-3 PUFA and an omega-6 PUFA, or a precursor or source thereof to the female during all or a portion of the pregnancy and lactation, to decrease the risk that the fetus will be born with or develop after birth a Reelin deficiency or dysfunction, wherein the administration of the PUFA is increased if the fetus is a male as compared to if the fetus is a female.

80. A method to prevent, delay the onset of, or reduce a symptom or disorder associated with Reelin deficiency or dysfunction in a child, comprising:
a) measuring the expression or biological activity of Reelin in a biological sample from the child; and
b) administering to the child a polyunsaturated fatty acid (PUFA) selected from the group consisting of an omega-3 PUFA and an omega-6 PUFA, or a precursor or source thereof, wherein the amount of PUFA administered is determined based on the measurement of expression or biological activity of the Reelin in the sample.

81. The method of claim 80, wherein the PUFA is provided in an infant formula supplemented with fatty acids comprising DHA and ARA.

82. The method of claim 80, wherein the PUFA is administered in an amount sufficient to compensate for reduced expression or activity of brain lipid binding proteins in the child or to improve the activity of brain lipid binding proteins in the child.

83. The method of claim 80, wherein the administration of the PUFA is sufficient to prevent, delay the onset of, or reduce the symptoms of autism.

84. The method of claim 80, wherein the administration of the PUFA is sufficient to prevent, delay the onset of, or reduce the symptoms of neuronal migration disorders.

85. A method to prevent, delay the onset of, or reduce a symptom of Alzheimer's disease associated with low molecular weight Reelin phenotypes, comprising:
a) identifying patients with Reelin deficiency or dysfunction, including patients with low molecular weight Reelin phenotypes; and
b) administering to the patient of (a) a polyunsaturated fatty acid (PUFA) selected from the group consisting of an omega-3 PUFA and an omega-6 PUFA, or a precursor or source thereof sufficient to compensate for the effects of Reelin deficiency or dysfunction in the patient.

86. A method to upregulate fatty acid binding proteins in a patient, comprising administering to a patient a polyunsaturated fatty acid (PUFA) selected from the group consisting of an omega-3 PUFA and an omega-6 PUFA, or a precursor or source thereof effective to upregulate FABP.

87. A method to upregulate Reelin expression or activity in a patient, comprising administering to the patient a polyunsaturated fatty acid (PUFA) selected from the group consisting of an omega-3 PUFA and an omega-6 PUFA, or a precursor or source thereof effective to upregulate Reelin expression or activity.

88. A method to improve neuronal migration in a patient, comprising administering to the patient a polyunsaturated fatty acid (PUFA) selected from the group consisting of an omega-3 PUFA and an omega-6 PUFA, or a precursor or source thereof effective to improve neuronal migration in the patient.

89. The method of claim 88, wherein neuronal migration is measured by measuring levels of Reelin expression or activity in the patient.

90. The method of claim 88, wherein neural function is measured by imaging techniques, and phenotypic evaluation.

91. A method to identify neural progenitor cells, comprising detecting Reelin expression or biological activity in a population of cells, wherein a defined level of Reelin expression or biological activity is associated with neural progenitor cells.

92. The method of claim 91, further comprising selecting the neural progenitor cells for which Reelin expression or biological activity was detected.

93. A method to monitor neural development, comprising:
a) providing a population of cells comprising neural progenitor cells;
b) detecting Reelin expression or activity in the population of cells;
c) exposing the population of cells to conditions under which the neural progenitor cells will develop into differentiated neural cells; and
d) monitoring the expression or activity of Reelin in the cells after step (c), to evaluate the development of the neural progenitor cells into differentiated neural cells.

94. The method of claim 93, further comprising contacting the population of cells of step (a) with a putative developmental regulatory compound prior to or concurrent with step (b), and determining whether the putative regulatory compound affects the development of the neural progenitor cells into differentiated neural cells by detecting Reelin expression or activity in the population of cells.

95. A method to treat or prevent a disorder associated with a deficiency or dysfunction in fatty acid binding proteins, comprising:
a) identifying patients with decreased expression or activity of at least one fatty acid binding protein; and
b) administering to the patient a polyunsaturated fatty acid (PUFA) selected from the group consisting of an omega-3 PUFA and an omega-6 PUFA, or a precursor or source thereof in an amount that is determined be sufficient to compensate for the effects of the decreased expression or activity of the fatty acid binding protein.

96. The method of claim 95, wherein the fatty acid binding protein is a brain lipid binding protein (BLBP).

97. The method of claim 95, wherein the fatty acid binding protein is a fatty acid binding protein in the heart.

98. A method to treat or prevent a disorder associated with reduced activity or dysfunction of a receptor for a fatty acid binding protein, comprising:
a) identifying patients with reduced activity or dysfunction of a receptor for a fatty acid binding protein; and
b) administering to the patient a polyunsaturated fatty acid (PUFA) selected from the group consisting of an omega-3 PUFA and an omega-6 PUFA, or a precursor or source thereof in an amount that is determined be sufficient to compensate for the effects of the reduced activity or dysfunction of a receptor for a fatty acid binding protein.

99. A pharmaceutical composition comprising an amount of a polyunsaturated fatty acid (PUFA) selected from the group consisting of an omega-3 PUFA and an omega-6 PUFA, or a precursor or source thereof, with at least one therapeutic compound for treatment or prevention of a disorder associated with Reelin deficiency sufficient to compensate for the reduced expression or activity of fatty acid binding proteins in a patient that has or is at risk of developing a Reelin deficiency.

100. The pharmaceutical composition of claim 99, wherein the therapeutic compound is a thyroid medication.

101. A method to diagnose a DHA deficiency in a patient, comprising:
a) measuring Reelin expression or biological activity in a biological sample from a patient;
b) comparing the Reelin expression or biological activity in the biological sample to a baseline level of Reelin;
c) measuring thyroid stimulating hormone (TSH) expression or biological activity in a biological sample from a patient;
d) comparing the TSH expression or biological activity in the biological sample to a baseline level of TSH; and,
e) making a diagnosis of the patient, wherein detection of a difference in the level of Reelin expression or biological activity in the biological sample as compared to the baseline level of Reelin expression or biological activity, and wherein detection of a difference in the level of TSH expression or biological activity in the biological sample as compared to the baseline level of TSH expression or biological activity, indicates a positive diagnosis of DHA deficiency in the patient.

102. The method of claim 101, wherein the biological sample is selected from the group consisting of a cell sample, a tissue sample, and a bodily fluid sample.

103. The method of claim 101, wherein the patient is pregnant or suspected of being pregnant.

104. A method to supplement PUFAs in a female during pregnancy and lactation, comprising:
a) measuring the expression and biological activity of Reelin in a biological sample from the mother of a fetus or child;
b) measuring the expression or biological activity of thyroid stimulating hormone in the biological sample;
c) administering a polyunsaturated fatty acid (PUFA) selected from the group consisting of an omega-3 PUFA and an omega-6 PUFA, or a precursor or source thereof to the mother of the fetus or child, wherein the amount of PUFA administered is determined based on the measurement of expression or biological activity of the Reelin in the sample from the parent, wherein the PUFA supplements the PUFA in the female and her fetus or child; and
d) administering at least one thyroid medication to the mother of the fetus or child if the measurement of Reelin and thyroid stimulating hormone in the sample from the mother is determined to be low as compared to a baseline level of Reelin and thyroid stimulating hormone.

105. A method to diagnose a fetal neurodevelopmental disorder, comprising:
a) measuring Reelin expression or biological activity in an amniotic fluid sample from a fetus;
b) comparing the Reelin expression or biological activity in the sample to a baseline level of Reelin; and,
c) making a diagnosis of the fetus, wherein detection of a difference in the level of Reelin expression or biological activity in the sample as compared to the baseline level of Reelin expression or biological activity, indicates a positive diagnosis of a neurodevelopmental disorder in the fetus.

106. The method of claim 105, wherein a fetus having a positive diagnosis in (c) is administered an amount of Reelin or reelin gene in utero sufficient to treat the neurodevelopmental disorder.

107. The method of claim 105, wherein a fetus having a positive diagnosis in (c) is administered an amount of Reelin postnatally sufficient to treat the neurodevelopmental disorder.

108. The method of claim 107, wherein the Reelin is administered in an infant formula.

109. A nutritional supplement or oral pharmaceutical, comprising an amount of Reelin sufficient to delay or prevent the development of a Reelin-deficiency or dysfunction or a disease or condition related thereto.

110. The nutritional supplement or oral pharmaceutical of claim 109, wherein the supplement is provided in infant formula.

111. The nutritional supplement or oral pharmaceutical of claim 109, wherein the supplement is provided to an infant by milk produced by the infant's mother, wherein the mother of the infant is supplemented with Reelin prior to or during lactation.

112. The method of claim 1 wherein the PUFA is a highly unsaturated fatty acid (HUFA).

113. The method of claim 1 wherein the PUFA is selected from the group consisting of arachidonic acid (ARA), eicosapentaenoic acid (EPA), docosahexaenoic acid (DHA) and docosapentaenoic acid (DPA).

114. The method of claim 1 wherein the PUFA is selected from the group consisting of ARA, EPA, and DHA.

115. The method of claim 1 wherein the PUFA is DHA.

116. The method of claim 1 wherein the source of the PUFA is selected from the group consisting of: fish oil, marine algae, and plant oil.

117. The method of claim 1 wherein the PUFA is DHA and wherein the precursor of DHA is selected from the group consisting of: α-linolenic acid (LNA), eicosapentaenoic acid (EPA), docosapentaenoic acid (DPA), and blends of precursors selected from the group consisting of LNA, EPA, and DPA.

118. The method of claim 1 wherein the PUFA is administered in a form selected from the group consisting of: a highly purified algal oil comprising the PUFA in triglyceride form, triglyceride oil comprising the PUFA, phospholipids comprising the PUFA, a combination of protein and phospholipids comprising the PUFA, dried marine microalgae, sphingolipids comprising the PUFA, esters, a free fatty acid, a conjugate of the PUFA with another bioactive molecule, and combinations thereof.

119. The method of claim 118, wherein the bioactive molecule is selected from the group consisting of a protein, an amino acid, a drug, and a carbohydrate.

120. The method of claim 1 wherein the PUFA is administered orally.

121. The method of claim 1 wherein the PUFA is administered as a formulation comprising the PUFA or precursor or source thereof selected from the group consisting of: chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods and combinations thereof.

122. The method of claim 121, wherein the PUFA in the formulation is provided in a form selected from the group consisting of: a highly purified algal oil comprising the PUFA, triglyceride oil comprising the PUFA, phospholipids comprising the PUFA, a combination of protein and phospholipids comprising the PUFA, dried marine microalgae comprising the PUFA, sphingolipids comprising the PUFA, esters of the PUFA, free fatty acid, a conjugate of the PUFA with another bioactive molecule, and combinations thereof.

123. The method of claim 1 wherein the PUFA is administered in a dosage of from about 0.05 mg of the PUFA per kg body weight of the patient to about 200 mg of the PUFA per kg body weight of the patient.
